(19) United States
(12) Patent Application Publication
(10) Pub. No.: US 2026/0360528 A1
(43) Pub. Date: Nov. 12, 2026

(54) SYSTEMS AND METHODS FOR CONTINUOUS PHYSIOLOGICAL PROFILING FROM IRREGULARLY SAMPLED, MULTI-SOURCE PHYSIOLOGICAL DATA WITH MISSING TIMEPOINTS

(71) Applicant: CALIFORNIA INSTITUTE OF TECHNOLOGY, Pasadena, CA (US)

(72) Inventors: Samuel SOLOMON, Rockville, MD (US); Wei Gao, San Marino, CA (US)

(21) Appl. No.: 19/673,967

(22) Filed: May 11, 2026

Related U.S. Application Data
(60) Provisional application No. 63/802,807, filed on May 9, 2025.

(51) Int. Cl. G16H 40/63

(52) U.S. Cl. CPC G16H 40/63

(57) ABSTRACT

Systems and methods for continuous physiological profiling and emotion-related decision support using a trained machine-learning model that defines an invertible mapping between an observation space of multimodal physiological data and a latent space representing a unified subject profile. The approach ingests physiological time-series data from one or more sources and generates the subject profile notwithstanding real-world acquisition artifacts including missing portions, irregular sampling, and temporal misalignment across modalities. The invertible formulation enables incorporation of additional observations over time by mapping new measurements into latent-space locations consistent with the existing profile, thereby updating the profile without full synchronization or complete datasets, and optionally reconstructing missing data. The subject profile supports downstream inferences including determination of an emotional state and initiation of an action, such as controlling delivery of a stimulus. Wearable sensor patches are exemplary data sources.

### CROSS-REFERENCE TO RELATED APPLICATIONS

This application claims the benefit of U.S. Provisional Application No. 63/802,807 filed on May 9, 2025, the contents of which are incorporated herein by reference in their entirety.

### STATEMENT REGARDING FEDERALLY SPONSORED R&D

This invention was made with government support under Grant No. W911NF-23-1-0041 awarded by Army Research Office, Grant No. N00014-21-1-2483 and N00014-21-1-2845 awarded by the Office of Naval Research. The government has certain rights in the invention.

### TECHNICAL FIELD

The present disclosure relates generally to machine-learning systems for processing multimodal physiological data, and in some implementations, may relate to geometrically structured, invertible architectures that generate and update a unified subject profile from irregularly sampled, temporally misaligned data, and/or partially missing physiological time-series data for downstream inference, including emotional state determination and management actions.

### BACKGROUND

Wearable and mobile computing devices have increased the availability of physiological measurements outside of controlled clinical settings. Sensors integrated into patches, watches, headsets, garments, and other form factors can capture signals indicative of autonomic and central nervous system activity, such as electrical potentials, electrodermal responses, temperature, motion, and other biomarkers. These measurements are increasingly used to characterize a user's physical and cognitive condition during daily activities.

Alongside advances in sensing hardware, there has been growing interest in “affective computing” and related technologies that seek to infer emotion-related states, stress, fatigue, arousal, engagement, or other psychophysiological conditions from physiological data. Such inferences may be valuable for mental health support, human-computer interaction, training and performance optimization, safety monitoring, and personalized wellness applications.

In practice, physiological monitoring systems frequently rely on multiple sensors or data sources that operate at different sampling rates, have different signal qualities, and are subject to different environmental and user-dependent artifacts. Real-world use introduces motion, perspiration, intermittent contact, electromagnetic interference, and device placement variability, each of which can affect signal quality and continuity.

A persistent challenge in multi-sensor systems is that physiological data streams are often incomplete or irregular. Samples may be dropped due to wireless transmission issues, battery-saving duty cycles, memory limits, or sensor saturation. Different devices may not share a common time base, leading to temporal offsets, drift, and misalignment between modalities. As a result, datasets collected “in the wild” can be fragmented and difficult to synchronize.

Conventional data-processing pipelines typically address these issues through preprocessing steps such as filtering, resampling, interpolation, imputation, and explicit temporal alignment. These steps can be brittle, require manual parameter tuning, and may introduce bias or distort the underlying physiology, particularly when the missingness is non-random or when signals are sparse, noisy, or subject-specific.

Machine-learning approaches have been applied to infer latent physiological or behavioral states from multimodal sensor inputs, including supervised classification and regression models, recurrent neural networks, attention-based architectures, and other sequence-processing methods. While such models can achieve high performance under controlled conditions, they may degrade when deployed on heterogeneous datasets that differ in sensor type, placement, sampling frequency, or population characteristics, raising concerns about generalization across devices, contexts, and users.

Interpretability and reliability are also important in physiological inference systems. Many high-capacity models operate as black boxes, making it difficult to explain how particular input signals contributed to an output state estimate or recommendation. This can hinder clinical adoption, reduce user trust, complicate debugging, and create challenges for safety-critical applications where confidence measures and failure detection are desirable.

There is also an increasing need for frameworks that represent physiological information in a unified manner across modalities and sources. Rather than treating each signal as an independent input channel, unified representations can support transfer across datasets, incremental incorporation of new sensor types, and downstream tasks beyond a single pre-defined prediction objective. Such representations may also facilitate personalization by capturing stable subject-specific characteristics alongside time-varying state information.

In some applications, physiological state inference is coupled to closed-loop interventions, in which a system adjusts feedback or stimuli in response to detected conditions. Examples include altering audio content, changing thermal or haptic outputs, modifying visual environments, delivering alerts, or adapting interactive experiences. Designing robust closed-loop systems often requires low-latency inference, stability under noisy measurements, and safeguards to avoid undesirable feedback dynamics.

Furthermore, practical deployment considerations can constrain the design of physiological inference systems. Power, compute, and memory limitations may motivate on-device processing, while privacy and security concerns may limit transmission of raw physiological data. Conversely, cloud or edge processing can enable more complex models but may introduce latency and connectivity dependence. These tradeoffs motivate continued development of physiological data processing techniques that are robust, efficient, and adaptable to real-world conditions.

### BRIEF SUMMARY

Various embodiments of the present disclosure may discuss a computer-implemented method for monitoring and managing emotional states in which multimodal physiological data associated with a subject is received from one or more data sources, the data may be processed using a trained model that defines an invertible mapping between an observation space and a latent space. A subject profile may be generated using the invertible mapping despite one or more data acquisition artifacts. An emotional state may be determined based on the subject profile, and an action may be caused based on the determined emotional state.

Various embodiments of the present disclosure may further discuss a computer-implemented method for monitoring and managing emotional states in which an initial subject profile is generated in a latent space based on a first portion of multimodal physiological data. Additional multimodal physiological data associated with the subject may be received after generating the initial subject profile. The additional data may include at least one of temporal misalignment with the first portion or missing portions. The additional data may be mapped into the latent space to associate the additional data with latent-space locations of the subject profile and to update the subject profile. An emotional state may be determined based on the updated subject profile, and an action may be caused based on the determined emotional state.

Various embodiments of the present disclosure may further discuss a computer-implemented method for generating and updating a unified subject profile from multimodal physiological data in which an initial subject profile is generated in a latent space from a first portion of the multimodal physiological data. Additional multimodal physiological data may be received after generating the initial subject profile. An invertible mapping may be applied to associate the additional data with corresponding latent-space locations, and the updated subject profile may be output for use in at least one downstream process.

Various embodiments of the present disclosure may further discuss a system for monitoring and managing an emotional state that includes one or more physiological sensors that generate multimodal physiological data, a stimulus device that delivers a stimulus to a subject, and a controller with one or more processors and memory storing instructions that cause receipt of the data, processing of the data using a trained model that defines an invertible mapping between an observation space and a latent space representing a subject profile, generation of the subject profile, determination of an emotional state based on the subject profile, and control of the stimulus device based on the determined emotional state.

Further embodiments may include receiving first multimodal physiological data to generate an initial subject profile and thereafter receiving additional physiological data, where the invertible mapping is used to map the additional multimodal physiological data into the latent space to update the subject profile without requiring temporal alignment between the additional multimodal physiological data and the first multimodal physiological data.

Further embodiments may include a trained model that includes a geometrically structured neural architecture that performs or represents transformations on a manifold, where processing includes applying an exponential map to map between the latent space and the observation space, and where processing further includes applying a plurality of exponential-map-based transformations in respective layers or subspaces of the trained model.

Further embodiments may include an invertible mapping that is bijective between the observation space and the latent space, and may further include generating the subject profile and updating the subject profile while reconstructing at least a portion of the multimodal physiological data corresponding to missing portions.

Further embodiments may include receiving multimodal physiological data that is generated by a plurality of wearable devices associated with the subject, and may include receiving multimodal physiological data that comprises time-series data having at least one of missing portions, irregular sampling, or temporal misalignment between modalities.

Further embodiments may include causing performance of the action by causing delivery of a stimulus to the subject based on the determined emotional state, where the stimulus may include at least one of an auditory stimulus, a thermal stimulus, a visual stimulus, or an immersive visual stimulus.

Further embodiments may include, for unified subject profile generation and updating, an invertible mapping that is bijective, a trained model that represents the invertible mapping as a composition of manifold-based transformations, mapping between the latent space and the observation space using an exponential-map-based parameterization, and an invertible architecture including at least one volume-preserving transformation, where associating additional multimodal physiological data includes applying an inverse of the invertible mapping.

Further embodiments may include mapping physiological data from a plurality of different modalities into a shared latent representation forming at least part of the subject profile, and may include multimodal physiological data that comprises time-series data having at least one of missing portions, irregular sampling, or temporal misalignment between modalities, including receiving observed physiological data from a plurality of physiological sensing modalities of one or more sensing devices.

Further embodiments may include, for the system, one or more physiological sensors carried by a skin-conformal wearable patch comprising a sweat-permeable substrate, where the sweat-permeable substrate includes a porous layer defining a plurality of pores or apertures extending between a skin-facing surface and an outward-facing surface of the wearable patch.

Further embodiments may include, for the system, multimodal physiological data that comprises time-series data having at least one of missing portions, irregular sampling, or temporal misalignment between modalities, and may include a wearable patch having conductive traces on the sweat-permeable substrate that electrically couple the one or more physiological sensors to an electrical interface and having a skin-adhesive layer on the skin-facing surface of the wearable patch.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein.

### BRIEF DESCRIPTION OF THE SEVERAL VIEWS OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1A is a diagram depicting an example real-time emotion monitoring and modulation architecture, in accordance with various embodiments of the presently disclosed technology.

FIG. 1B is a diagram depicting an example wearable patch, in accordance with various embodiments of the presently disclosed technology.

FIG. 1C is a diagram depicting an example therapy method selection and adaptive intervention strategy, in accordance with various embodiments of the presently disclosed technology.

FIGS. 2A-B are diagrams depicting exemplary Lie manifold neural architectures for biomarker reconstruction and health profile generation, in accordance with various embodiments of the presently disclosed technology.

FIGS. 3A-B are diagrams depicting an example schematic of a phase-separated float assembly (PSFA) technique, in accordance with various embodiments of the presently disclosed technology.

FIG. 3C is a diagram depicting an example schematic of light-emitting diode (LED) arrays fabricated with strain resilient interconnects, in accordance with various embodiments of the presently disclosed technology.

FIG. 4A is a diagram depicting an example schematic for a wearable patch platform for monitoring biomarkers and providing heat therapy, in accordance with various embodiments of the presently disclosed technology.

FIG. 4B is a block diagram depicting an example wireless wearable system and digital processing pipeline that can interface with the wearable patch platform, in accordance with various embodiments of the presently disclosed technology.

FIG. 4C is a diagram depicting various exemplary physiological responses and features across sensing platforms and/or conditions, in accordance with various embodiments of the presently disclosed technology.

FIG. 5 is a diagram depicting an example schematic of an emotion modulation therapy algorithm, in accordance with various embodiments of the presently disclosed technology.

FIGS. 6A-B are diagrams depicting various exemplary health profile distributions, confusion matrices, therapy effectiveness comparisons, and emotion modulation effects, in accordance with various embodiments the presently disclosed technology.

FIG. 7 is a diagram depicting an example wearable patch platform fabrication process, in accordance with various embodiments of the presently disclosed technology.

The figures are not intended to be exhaustive or to limit the invention to the precise form disclosed. It should be understood that the invention can be practiced with modification and alteration, and the disclosed technology be limited only by the claims and the equivalents thereof.

### DETAILED DESCRIPTION

Embodiments of the present disclosure may discuss techniques for transforming heterogeneous physiological measurements into a unified, continuously updateable subject profile, where aspects of the technical embodiments may include a trained model that can define an invertible relationship between observed physiological data and a latent representation. The latent representation may function as a persistent profile that can be refined over time and used to support downstream decisions, including emotion-related monitoring, state inference, and management actions.

Physiological monitoring in realistic settings often produces data that is difficult to use with conventional pipelines. Wearable sensors and other sensing devices can generate multiple streams that vary in sampling rate, time base, quality, and availability. A head-worn device may produce high-rate electrical signals, a wrist device may produce lower-rate optical or motion features, and a skin patch may produce electrodermal and temperature measurements. These streams can arrive asynchronously and can be intermittently absent due to battery duty cycling, wireless transmission loss, sensor contact changes, motion artifacts, and environmental interference.

Conventional inference systems frequently assume that inputs are well-formed, synchronized, and present in a fixed schema. Many models rely on a forward pass that expects a consistent vector of features at each time step. In practice, engineers address this mismatch by imposing preprocessing steps that resample signals onto a shared grid, align devices by timestamps, and impute missing values. These steps can be brittle, can introduce artifacts, and often require modality-specific heuristics that do not transfer well across devices, subjects, and datasets.

The disclosed approaches reframe the problem around a subject profile maintained in a latent space and an invertible mapping that links the latent space to an observation space. The observation space may correspond to the physiological measurements as they are obtained or represented, including raw samples, windowed features, and metadata such as modality identifiers and time information. The latent space may correspond to a unified representation that “explains” the observed signals and aggregates information across modalities and across time.

Invertibility may be used to support reasoning in both directions. Mapping from observations into the latent representation may support inference and profile construction from measured data. Mapping from the latent representation back into the observation space may support reconstruction and consistency checks because the model can generate predicted observations that should match the measurements that were actually observed. This bidirectionality is distinct from typical black-box predictors that often learn a one-way mapping from inputs to outputs and that cannot naturally support inversion.

A practical benefit of the invertible formulation is that it may support building a coherent profile even when observed data is fragmented, temporally misaligned, irregularly sampled, or partially missing. The system does not need to wait for a complete synchronized “frame” of inputs before it can update the profile. Instead, each measurement or measurement window can be treated as an independent constraint that must be consistent with the same underlying subject profile.

This viewpoint can be explained in plain language using a “painting” analogy. The subject profile may be analogous to a partially completed canvas. Different artists may arrive at different times and paint different parts, sometimes with different styles or tools. The system's role is to determine where each new contribution belongs within the overall painting and to integrate it without requiring that every artist paint simultaneously or that each artist covers the entire canvas. The profile becomes more complete and more reliable as additional constraints arrive.

The constraint-based integration is especially relevant for multi-device deployments. Different devices may not share a clock, and their sampling frequencies may differ. Rather than forcing a global alignment step, the model can incorporate time information as part of the representation and can map new observations into latent-space locations that best match the current state of the profile. When the incoming observation corresponds to a latent region that has been previously constrained, the profile can be refined and the system's confidence at that region can increase. When the incoming observation corresponds to a region that has not been constrained, the profile can be extended or filled in.

Aspects of these implementations may exhibit incremental behavior by reciting generation of an initial subject profile from a first portion of data, followed by receipt of additional data and mapping of that additional data into the latent space to update the profile. This incremental update may be intended to capture continuous operation where new physiological measurements may arrive over time and the system may continuously integrate them, without requiring full temporal alignment to the original data used to seed the profile.

The invertible mapping may be bijective over a modeled domain, meaning that the mapping may be one-to-one and onto between the observation representation and the latent representation. Bijectivity may be valuable because it supports interpretability and stable inference. If a particular downstream conclusion is reached, the system can trace which latent factors and which observed constraints drove that conclusion. If the system reconstructs or forecasts signals, the reconstructed output can be mapped back to latent factors in a way that preserves correspondence rather than collapsing multiple inputs into an ambiguous output.

The trained model can be described as geometrically structured because its internal transformations may be constrained to follow geometric principles rather than arbitrary unconstrained matrix operations. This structure can be implemented by representing transformations on a manifold. The manifold perspective may provide a way to encode transformations that preserve important properties of signals as they move through hidden layers, such as normalization properties and well-behaved reversibility.

Lie manifold and Lie group based transformations may be utilized herein. A Lie group may provide a structured set of transformations with group properties, including composition and inversion. A Lie manifold may provide the geometric space on which these transformations act. Implementations based on these concepts can ensure that transformations remain within a desired class, such as generalized rotations, across training and inference.

The exponential map may be relevant in connecting learnable parameters to structured transformations on a Lie group. The exponential map can take a parameterization in a local linear space, such as a tangent space, and map it onto the manifold in a way that yields a valid group element. This is technically relevant because it may constrain learning so that updates do not drift into invalid or ill-conditioned transformations.

Examples may include the exponential map of a skew-symmetric matrix. Exponentiating a skew-symmetric matrix may yield a rotation-like operator that is inherently reversible, since rotating “the other way” corresponds to applying the inverse. Such transformations may be normalization-preserving in the sense that they do not arbitrarily scale the representation, which contributes to stability during training and inference.

Volume preservation may also be associated with rotation-like transformations. When a transformation is volume-preserving, its Jacobian determinant may be one, meaning it does not expand or compress volume in the transformed space. This property can simplify training because the transformation may avoid runaway amplification or collapse in hidden representations. At the same time, the architecture can include reversible nonlinear subspace transformations that allow the volume to transform slightly while retaining invertibility. This supports expressiveness without giving up the core reversible structure.

The system may use multiple exponential-map transformations, not a single transformation between input and output. Multiple exponential-map blocks can be applied across layers or across distinct nonlinear subspaces. This allows the model to represent complex relationships among modalities and time while maintaining a principled geometric structure at each stage. The result may be a deep architecture that remains invertible and can be traversed forward and backward.

The stability benefits of structured transformations may be relevant to training dynamics. Many deep learning systems suffer from vanishing gradients or exploding gradients when the internal transformations amplify or shrink representations unpredictably across layers. Rotation-like, normalization-preserving transformations may provide a controlled way to move representations through depth. This can improve numerical stability and support training with practical learning rates and long training horizons.

Certain embodiments may discuss a training perspective that differs from conventional forward-only modeling. The invertible formulation may enable the system to treat the profile as a learnable output that must explain observed constraints, which can be described as solving a simpler inverse problem. Rather than requiring that a fixed set of synchronized inputs produce the profile, the system can start from a well-formed latent representation and learn how that representation generates observed signals through invertible transformations. Because the mapping is invertible, the trained model can still be used in the forward direction for inference from observed data to latent profile.

This viewpoint supports a decoder-oriented training process in which the system can learn to generate plausible physiological observations from latent profile states while maintaining invertibility. Each observed timepoint can be treated as a constraint that may be satisfied. As additional observations arrive, the model can update the latent profile to better satisfy the growing set of constraints. This helps the system remain stable under different temporal fragmentations and under different patterns of observation availability.

Adaptive behavior in the architecture can be enabled by the exponential formulation. When the learned generator for a rotation-like transformation becomes small, the exponential can yield an identity-like operation, since an exponential of zero corresponds to an identity element. This supports the ability to cull or deactivate transformations that become irrelevant during training, which can be viewed as an adaptive reduction in effective model complexity.

This adaptive behavior can be contrasted with conventional random dropout. Instead of randomly removing units, the model can learn that certain transformations should collapse to identity operations. This is particularly relevant when working with smaller datasets or when the model includes matrices with quadratic parameter counts relative to signal dimension. By learning to remove unnecessary transformations, the effective parameter count can be reduced during training while preserving invertibility and the structured nature of the remaining transformations.

The profile-based formulation may also be designed to generalize across datasets and across device configurations. Multiple datasets with different sensor types may be used and that the model may not require the presence of any specific modality. The system can treat any observed physiological measurement as an observation that can be incorporated into the profile. This supports the idea of a foundational model that can be adapted to new datasets without requiring that every dataset share the same sensor suite.

A practical consequence of modality agnosticism is that the profile can act as a common representation across different studies and different labs. One dataset may include electrodermal activity and temperature, another may include optical heart-related measures and motion, and another may include electrical brain-related measurements. The system can map each dataset's available observations into the latent profile space, which can provide a consistent basis for downstream inference, comparisons, personalization, and transfer.

Reconstruction may be an operational feature enabled by invertibility. When sensor data includes missing portions, the system can generate estimates of missing segments by mapping from the latent profile back into the observation space. Reconstruction can be used for data completion, denoising, artifact correction, and standardization. Large-scale reconstruction may be possible and the ability to reconstruct a substantial number of missing data points across a large set of biomarkers, which may be aligned with the goal of robust operation under realistic data fragmentation.

Forecasting and prediction can also be supported using the framework. Once the profile captures temporal structure, the system can estimate how a physiological trajectory may be expected to evolve and can generate predicted future observations or predicted future profile states. These capabilities may be useful for anticipating changes in downstream states, including affective states, and for supporting closed-loop actions that may be most effective when applied proactively.

Downstream inference may include determining an emotional state based on the subject profile. Emotional state can be expressed as a discrete class, a continuous score, a vector in an affective space, and/or a probability distribution. Because the subject profile integrates multi-modal and multi-source information, the emotional state determination can be made even when some modalities are absent, when data is sparse, or when data is not synchronized. The model's interpretability benefits can be leveraged to explain which observations contributed to the inferred state.

Actions based on the determined emotional state can be implemented in many ways, including triggering notifications, logging events, updating interfaces, or controlling external devices. Closed-loop management actions for such implementations may include delivering stimuli. Stimuli can include auditory cues, thermal stimulation, visual outputs, and immersive visual outputs. Control policies can be adaptive, meaning the stimulus parameters can change over time based on updated profile estimates and updated state determinations.

Wearable sensing hardware may provide a concrete and commercially relevant example of data acquisition. A skin-conformal patch can collect physiological signals while maintaining comfort and wearability. A sweat-permeable substrate can improve long-wear performance by reducing moisture buildup. A porous layer with pores or apertures can provide breathability while supporting stable sensor contact.

A patch implementation can include skin-contact sensors such as electrodes and thermal elements, conductive traces for interconnection, and an electrical interface to communicate with a controller or a local processor. An adhesive layer can maintain attachment to skin while allowing the patch to remain thin and flexible. The patch can serve as one of multiple data sources feeding the unified profile, and it can be combined with other wearable devices without requiring that all devices share a common clock or data format.

Compute placement is not necessarily central, and the disclosure indicates that the model can run on-device or off-device. The trained model can be small enough to execute on a wearable controller for low latency and privacy. It can also execute on a mobile device or a remote server when additional compute resources are desired. The “computer-implemented” framing reflects that at least one processor executes the mapping and profile update steps, rather than imposing any specific architecture.

Overall, the detailed operation can be understood as a continuous loop. Physiological observations may be received from one or more sources. The trained invertible model maps observations into a latent profile representation and updates that profile as new observations arrive. The profile supports reconstruction and standardization of signals and supports downstream inference such as emotional state determination. The system then causes an action, which can include stimulus delivery, and continues integrating new physiological observations to refine the profile and adapt downstream outputs in real time.

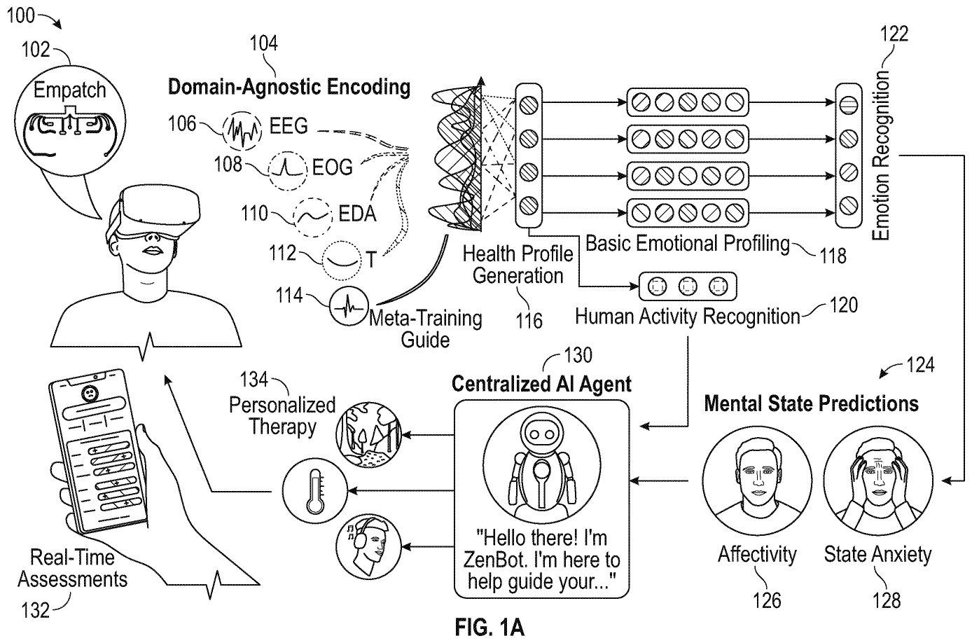

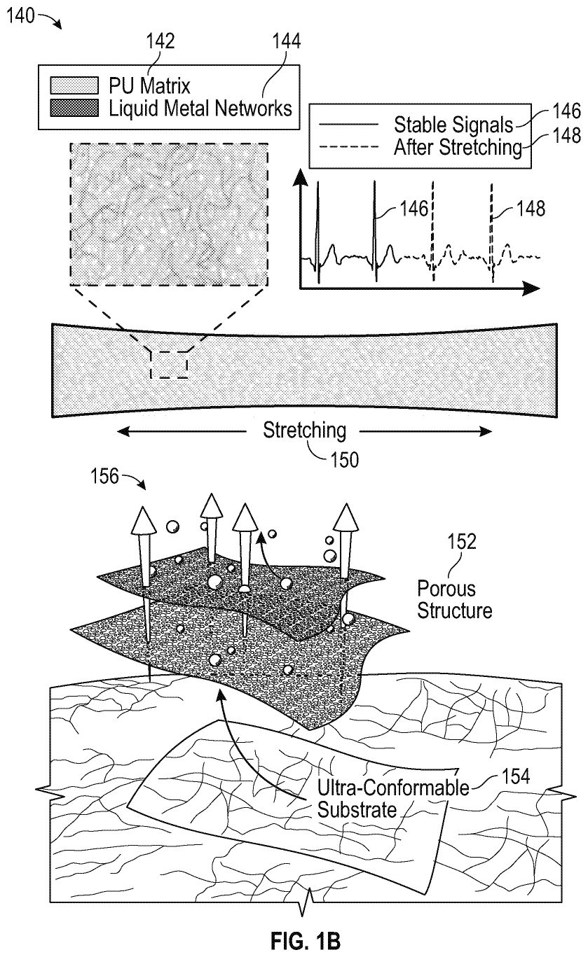

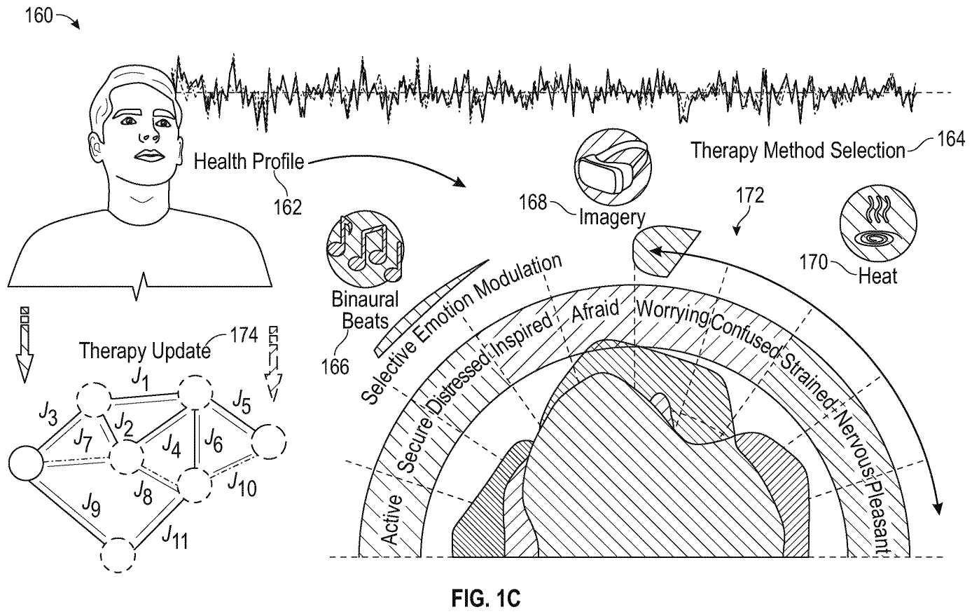

Turning now to the figures, FIG. 1A is a diagram depicting an example real-time emotion monitoring and modulation architecture, in accordance with various embodiments of the presently disclosed technology. In the illustrated example, architecture 100 is shown as an end-to-end framework for acquiring multimodal physiological information, generating a unified subject-level representation, producing downstream inferences, and driving real-time actions. The architecture may be implemented as part of, for example, an EMPATCH platform 102, which may operate with one or more wearable devices and one or more computing systems to support continuous physiological profiling and emotion-related assessments. In various embodiments, the platform may be embodied as a conformal, wearable electronic system, such as the EMPATCH platform described in greater detail in connection with other figures, that continuously and simultaneously monitors multiple physiological modalities while the subject goes about normal daily activities, thereby enabling persistent, real-time characterization of the subject's physiological and affective state without requiring laboratory-controlled conditions or manual data synchronization.

The architecture 100 may include a domain-agnostic encoding component 104 that receives multimodal physiological data from heterogeneous sources. Example input modalities can include electroencephalography (EEG) data 106, electrooculography (EOG) data 108, electrodermal activity (EDA) data 110, and skin temperature data 112, though the architecture is not limited to these particular modalities. Each of these signals may reflect distinct but complementary aspects of autonomic and central nervous system activity. EEG measures extracellular voltage fluctuations arising from synchronized postsynaptic activity in cortical neurons, and frontal EEG asymmetry in particular has been implicated in emotional processing, reflecting dynamic interplay between brain activity and affective state. EOG captures changes in the corneo-retinal potential associated with eye movements, including saccades, fixations, and blinks, which correlate with cognitive load and affective arousal. EDA reflects changes in skin conductance driven by sympathetic nervous system activity and serves as a marker of autonomic emotional arousal. Skin temperature, regulated by the autonomic nervous system, provides an additional indicator of emotional and thermoregulatory responses that can distinguish states such as relaxation or stress-induced vasoconstriction.

Domain-agnostic encoding 104 may be designed to ingest these inputs in a manner that does not require a fixed sensor suite, does not require that all modalities be present at all times, and does not require strict temporal alignment, thereby accommodating real-world conditions such as irregular sampling, missing portions, and device-to-device timing differences. Each observed measurement may be treated as an independent, time-dependent observable constraint on the underlying subject profile rather than as a component of a fixed, synchronized input vector, which allows the encoding component to accept any available combination of physiological signals at any given moment and incorporate them into a unified representation without discarding or waiting for missing channels.

The architecture 100 may further include a meta-training guide 114 that supports learning and generalization across different datasets, sensing configurations, and usage contexts. In practice, physiological data collected across different studies, platforms, and populations can differ substantially in sensor type, body location, sampling frequency, and annotation strategy. For example, one dataset may contain frontal EEG and EDA signals from a head-worn device, while another may contain photoplethysmography, EDA, and skin temperature signals from a wrist-worn device, and yet another may contain chest and wrist measurements from separate concurrently worn devices.

The meta-training guide 114 may enable the trained model to learn shared affective and physiological patterns that generalize across these heterogeneous configurations, such that the resulting latent representation remains consistent and meaningful regardless of which combination of modalities or datasets is encountered at inference time. This approach can support a foundational modeling framework in which diverse datasets spanning varied platforms, body locations, biomarkers, and emotional annotation strategies can all be incorporated into a common representational framework without requiring bespoke retraining for each new device configuration or study population. By reducing dataset-specific parameters while preserving shared structure, the meta-training process can help the model generalize to new datasets, including out-of-domain configurations, with limited additional data.

The outputs of domain-agnostic encoding 104, optionally informed by meta-training guide 114, may feed into health profile generation 116, which may produce a unified subject profile in a latent space. The trained model underlying health profile generation 116 may define an invertible mapping between the observation space, which corresponds to the multimodal physiological data as acquired, and the latent space, which corresponds to a unified representation that explains the observed signals and aggregates information across modalities and time. The model may be trained in a reverse, decoder-oriented manner in which the latent profile may be learned as the output that must account for all observable physiological signals, treating each recorded timepoint as a constraint to be satisfied.

Because the mapping is invertible, the model can also be traversed in the forward direction at inference time, mapping newly observed physiological measurements back into latent-space locations consistent with the evolving profile. In practice, the subject profile can be formed and maintained even when only partial observations are available, and it can be incrementally refined as new observations arrive by associating each new measurement with the corresponding region of the latent space, thereby increasing confidence at that location and progressively completing the profile without requiring complete, synchronized data across all modalities. The invertible architecture may employ geometrically structured transformations, such as those based on Lie group and Lie manifold principles, in which learnable parameters are expressed through matrix exponentials of skew-symmetric generators, yielding rotation-like transformations that are inherently reversible, normalization-preserving, and geometrically interpretable. Multiple such transformations may be applied across layers or distinct nonlinear subspaces of the architecture, supporting expressiveness while maintaining the principled geometric structure and bidirectional traversability of the overall mapping. A practical consequence of this formulation is that the health profile generation component can reconstruct estimates of missing physiological segments by mapping from the latent profile back into the observation space, supporting data completion, artifact correction, and signal standardization alongside inference.

The unified profile produced by health profile generation 116 may support multiple downstream inference tasks simultaneously, operating from the same continuously updated latent representation without requiring separate preprocessing pipelines for each task. The illustrated example includes basic emotional profiling 118, human activity recognition 120, and emotion recognition 122, any of which may operate on the profile directly or on standardized representations derived from the profile. Basic emotional profiling 118 may extract shared emotion primitives from the latent profile that serve as building blocks for characterizing affective states, with bidirectional traceability from model outputs back to the individual physiological observations that contributed to them.

Human activity recognition 120 may operate concurrently to provide contextual awareness that can inform how physiological signals are interpreted, since the same physiological pattern may carry different affective significance depending on whether the subject is at rest, engaged in physical activity, or performing a cognitive task. Emotion recognition 122 may combine the extracted emotion primitives with subject-specific information from the profile to generate personalized emotion estimates that account for inter-individual variability in how physiological states map to affective experiences.

The architecture may also include mental predictions 124, which may generate higher-level assessments of mental and psychological state from the same or similar underlying profiles, including affectivity 126, such as positive and negative affect scores derived from instruments like the Positive and Negative Affect Schedule, and state anxiety 128, such as scores analogous to those captured by the State-Trait Anxiety Inventory. By supporting all of these downstream tasks from a single, continuously updated latent profile, the architecture avoids duplicative computation and enables cross-task consistency, where changes detected through emotion recognition and activity context jointly inform higher-level mental state predictions without requiring independent sensor pipelines for each output.

The architecture 100 may also include a centralized AI agent 130 that coordinates inference outputs and closes the loop with real-time response actions. The centralized AI agent 130 may receive and integrate the outputs of the downstream inference tasks, including the emotional profiling, activity recognition, and mental state predictions, and may apply an adaptive control policy to determine the most appropriate intervention given the subject's current assessed state, their prior affective history, and the effectiveness of previously delivered interventions. The centralized AI agent 130 may provide real-time assessments 132 based on the current subject profile and downstream task outputs, summarizing the subject's inferred affective and psychological condition in a form that can be used to guide therapeutic decision-making.

Based on these assessments, the centralized AI agent 130 may control or recommend personalized therapy 134 tailored to the subject's current and evolving state. Personalized therapy 134 may encompass a range of sensory intervention modalities, including auditory interventions such as binaural beats, which present sinusoidal tones at slightly different frequencies to each ear in order to entrain brainwave activity toward frequency bands associated with relaxed or focused mental states; thermal interventions such as targeted heat therapy, which activates thermoregulatory pathways and can promote parasympathetic activation and reduction of stress-related physiological arousal; visual interventions such as calming imagery; and immersive visual interventions delivered through virtual reality or holographic displays, which have been shown to produce particularly pronounced reductions in negative affect and state anxiety.

In various embodiments, the control policy governing therapy selection may be adaptive, updating intervention parameters in real time based on the measured physiological response to prior stimulation and the trajectory of the subject profile, so that the system converges toward an intervention state optimized for the individual subject rather than applying a fixed, population-level protocol. In this way, the architecture as a whole may support a closed-loop workflow in which multimodal physiological observations are continuously converted into an updateable latent profile, the profile supports real-time emotional, behavioral, and psychological assessments across multiple inference tasks simultaneously, and those assessments are used to drive adaptive, personalized interventions that are refined over time as the profile accumulates richer information about the subject.

FIG. 1B is a diagram depicting an example wearable patch, in accordance with various embodiments of the presently disclosed technology. The illustrated platform 140 may be implemented as a soft bioelectronic interface for capturing multimodal physiological signals during real-world use, including during motion, perspiration, and repeated mechanical deformation, while maintaining user comfort and stable skin contact over extended wear periods. In various embodiments, this platform may correspond to an instance of the EMPATCH device described herein, which may be applied conformally to the skin, such as on the forehead, to continuously record EEG, EOG, EDA, and skin temperature signals without requiring rigid housings, gel electrodes, or manual signal synchronization.

The illustrated platform 140 may be fabricated using a phase-separated float assembly process that produces an ultrathin, elastic, breathable, and sweat-permeable nanocomposite electronic skin. This fabrication process may be begin with a nanocomposite precursor solution in which polyurethane is dissolved in tetrahydrofuran and silver nanowires are dispersed in ethanol. When the precursor solution is drop-cast onto a water bath, the solvents dissolve and reduce surface tension, inducing Marangoni flow that expands the droplet boundary and forms an ultrathin film at the water-air interface. Concurrently, evaporation-driven liquid-liquid demixing induces phase separation, creating polyurethane-rich and polyurethane-poor regions into which the silver nanowires, due to their amphiphilic ligands and immiscibility with the polymer, preferentially localize. The result may be a randomly distributed, interconnected network of silver nanowires confined within a continuous porous polyurethane matrix that provides both mechanical compliance and electrically conductive percolation pathways. This process may enable rapid, scalable fabrication of large-area films exceeding 200 cm² with thicknesses on the order of approximately 1 micrometer for the porous polyurethane substrate layer and approximately 6 micrometers for the porous silver nanowire nanocomposite electrode layer, and with stretchability exceeding 400% strain.

The illustrated platform 140 may include a polymer substrate formed as a polyurethane (PU) matrix 142 that supports embedded conductive interconnects formed as liquid metal networks 144. The PU matrix 142 may serve as the primary structural and mechanical foundation of the device, providing elasticity, skin conformability, and a porous architecture that promotes breathability and moisture transport. The liquid metal networks 144 may be formed from a porous liquid metal composite and serve as the strain-resilient electrical interconnects that route signals from the on-skin sensing electrodes to the electrical interface of the device. Because the liquid metal composite may be both porous and intrinsically fluid, it may accommodate large mechanical deformations without the resistance changes that typically accompany strain in conventional metallic or non-porous conductive materials.

The porous liquid metal composite interconnects have been demonstrated to maintain consistent electrical performance through bending, twisting, and uniaxial stretching, and exhibit outstanding mechanical robustness, surviving aggressive mechanical insults including scalpel punctures, hammer impacts, and tweezer pricks without degradation of electrical performance. Cyclic stretching tests have indicated that the porous silver nanowire nanocomposite electrodes may sustain minimal resistance change, with a relative resistance ratio of approximately 4.8 after 3,000 stretching cycles at 25% strain, substantially outperforming non-porous control materials that exhibited resistance ratios exceeding 200 under the same conditions. This superior electromechanical stability may arise from synergistic stress dissipation within the elastic polyurethane matrix combined with the strain-adaptive geometry of the silver nanowire network.

Performance under mechanical strain may be depicted in the illustrated diagram, where the platform maintains stable signals 146 during and after stretching 148, 150. In this context, the PU matrix 142 may provide the elastic restoring force and mechanical support that allows the device to follow skin deformations without delaminating or cracking, while the liquid metal networks 144 provide conductive pathways that redistribute internal stresses and maintain electrical continuity under strain. The strain-invariant electromechanical properties of the liquid metal composite interconnects are particularly important in wearable physiological monitoring, where motion, facial expressions, and postural changes continuously subject the device to dynamic mechanical loading. By tolerating these deformations without introducing signal artifacts or open-circuit failures, the device can support reliable acquisition of electrophysiological signals across the full range of daily activities encountered in realistic monitoring scenarios.

The illustrated platform further may include a porous structure 152 and an ultra-conformable substrate 154 that together promote intimate skin contact while substantially improving breathability during long-duration wear. The porous structure 152 may be defined by a network of interconnected pores and apertures that extend between the skin-facing surface and the outward-facing surface of the device, forming pathways through which moisture vapor can freely pass. This architecture may be inspired by the sweat-pore structure of biological skin and enable the device to operate in a sweat-permeable 156 manner, meaning that perspiration generated at the skin surface can escape through the device rather than accumulating at the skin-device interface. Moisture buildup at the skin interface may be a significant practical barrier to long-duration wearable biosensing because trapped sweat can degrade electrode-skin impedance, reduce signal quality, cause skin irritation, and ultimately reduce user compliance during extended monitoring sessions. Water vapor permeability testing has indicated that the porous nanocomposite films may exhibit substantially higher moisture transmission rates compared to non-porous counterparts of equivalent thickness, and long-term biocompatibility may be confirmed through hematoxylin and eosin histological staining and fluorescent immunohistochemical analysis in animal models, demonstrating the absence of significant inflammatory response at the skin interface following extended application.

The ultra-conformable substrate 154 may result from the extreme thinness of the porous polyurethane layer, which allows the device to drape and conform to microscale surface features of skin without applying significant mechanical load or pressure, thereby minimizing motion artifacts arising from relative displacement between the electrode and the skin surface. The ultrathin porous electrode configuration may also achieve lower electrode-skin contact impedance compared to thicker electrode counterparts, due to the more intimate skin interface enabled by the conformal architecture, and EOG signals recorded by the porous on-skin electrodes before and after sweating have been shown to closely match those obtained with conventional silver-silver chloride gel electrodes in terms of signal fidelity and signal-to-noise ratio, confirming that the sweat-permeable porous architecture does not compromise electrophysiological recording quality during perspiration.

It should be understood that the wearable patch illustrated in FIG. 1B and described in connection with the EMPATCH platform represents one exemplary hardware implementation through which the physiological data acquisition capabilities of the presently disclosed system may be realized. The trained model, invertible mapping, subject profile generation and update logic, downstream inference components, and closed-loop action capabilities described throughout this disclosure are not limited to any particular sensing hardware, form factor, electrode material, substrate composition, or data acquisition modality. One of ordinary skill in the art will appreciate that the disclosed system may be implemented with a wide variety of physiological sensing configurations, including but not limited to rigid or flexible circuit boards, textile-integrated electrodes, gel-based clinical electrode arrays, optical sensing platforms such as photoplethysmography devices, inertial measurement units, acoustic sensors, implantable or near-body sensors, and any combination of on-body, near-body, or remote sensing modalities capable of generating physiological time-series data.

Similarly, the number, type, and placement of sensors may vary without departing from the scope of the disclosed technology, and the domain-agnostic encoding component of the architecture may be designed to accommodate such variation by treating each available physiological measurement as an independent observable constraint on the subject profile rather than requiring a fixed, predefined sensor suite. The description of the wearable patch and its associated fabrication techniques is therefore provided to illustrate one concrete and commercially relevant embodiment of the data acquisition layer of the system, and should not be read as limiting the scope of the claims to any particular hardware implementation.

FIG. 1C is a diagram depicting an example therapy method selection and adaptive intervention strategy, in accordance with various embodiments of the presently disclosed technology. The strategy shown generally at 160 may use a health profile 162 as the primary input to drive therapy method selection 164. The health profile 162 may represent a unified, continuously updated subject profile derived from multimodal physiological observations and maintained in the latent space defined by the invertible mapping described herein. In addition to encoding instantaneous state estimates, the health profile 162 may capture temporal trends in physiological and affective state, uncertainty measures reflecting the completeness and consistency of the underlying observations, and historical context accumulated over prior monitoring periods. This richness of representation may allow the therapy selection component to make decisions that are sensitive not only to the subject's current condition but also to the trajectory of that condition and to the degree of confidence warranted by the available data, supporting more nuanced and appropriate intervention choices than would be possible from a single-timepoint snapshot of physiological activity.

Therapy method selection 164 may include selecting among multiple therapy modalities, including binaural beats 166, imagery 168, and heat therapy 170, though the architecture is not limited to these particular modalities and may be extended to encompass additional sensory, behavioral, or pharmacological interventions as appropriate for the application context. Binaural beats 166 may involve the simultaneous presentation of pure sinusoidal tones at slightly different frequencies to each ear, producing a perceived auditory beat at the frequency difference between the two tones, which has been associated with entrainment of cortical oscillatory activity toward frequency bands corresponding to relaxed, focused, or meditative mental states, including alpha and theta band activity. Imagery 168 may encompass guided visualization or presentation of calming visual content that engages attentional and emotional processing pathways to modulate affective state, and may be delivered through conventional displays or through immersive virtual reality environments that produce more pronounced engagement with the presented visual content, the latter of which has been associated with particularly strong reductions in state anxiety and negative affect in prior work. Heat therapy 170 may involve targeted delivery of controlled thermal stimulation to the skin surface, which activates cutaneous thermoreceptors and thermoregulatory pathways and has been shown to promote parasympathetic nervous system activation, reduce physiological markers of stress-related arousal, and support subjective relaxation.

Selection logic governing the choice among these modalities may be rule-based, model-based, or hybrid in nature, and may incorporate individualized factors such as the subject's prior physiological response patterns to each modality, expressed user preferences, current activity context as determined by concurrent human activity recognition, contraindications arising from the subject's health profile, and specific target outcomes such as anxiety reduction, mood elevation, or arousal regulation. By treating the health profile 162 as a common representational substrate that integrates information across modalities and time, the therapy selection component can make consistent and well-grounded decisions even when the underlying sensor inputs are heterogeneous, partially available, or intermittently interrupted.

The portion identified generally at 172 illustrates an example intervention and feedback workflow in which a selected therapy modality may be delivered and evaluated over time. Once a therapy modality is selected and initiated, the system may continue to receive and integrate new physiological observations into the subject profile, thereby monitoring the subject's response to the intervention in real time. The feedback workflow may include comparing the trajectory of relevant profile features and downstream state estimates before and after the onset of intervention, assessing whether the desired direction of change in affective or physiological state is being achieved, and quantifying the magnitude and rate of the observed response relative to prior intervention episodes for the same subject. This continuous monitoring allows the system to detect both positive therapeutic responses, such as reductions in EDA reflecting decreased sympathetic arousal or frontal EEG asymmetry shifts consistent with improved affect, and null or adverse responses that may indicate that the current modality or parameterization is not well matched to the subject's current state. Based on these assessments, the system may determine whether to maintain the current modality and parameters, adjust one or more delivery parameters such as beat frequency, imagery content, or thermal intensity and spatial distribution, or transition to a different modality entirely, in each case grounding the decision in the updated health profile rather than in a fixed, time-invariant protocol.

The strategy may further include therapy update 174, which implements an adaptive update mechanism that modifies therapy selection and delivery on an ongoing basis as additional physiological data is received and incorporated into the health profile 162. Therapy update 174 may adjust any combination of intervention parameters, including intensity, duration, timing, sequencing, and modality choice, based on the evolving state of the health profile and on the accumulated history of observed outcomes across prior intervention episodes. In this manner, the intervention strategy can personalize over time to the individual subject, converging toward parameter configurations and modality sequences that reliably produce the desired affective outcomes for that subject specifically, rather than applying population-level defaults that may be suboptimal for individuals who differ in baseline physiology, responsiveness, or preference.

The adaptive update process may be directly enabled by the continuous, incremental profile update mechanism described throughout this disclosure, since each new physiological observation refines the profile and can immediately inform revised therapy decisions without requiring a complete resynchronization of the data pipeline or a full model retraining step. The result may be a closed-loop personalization system in which the intervention strategy and the physiological monitoring system co-evolve over the course of a monitoring session, with each new measurement simultaneously improving the accuracy of the subject's health profile and providing additional evidence that can sharpen the therapy selection and adaptation logic.

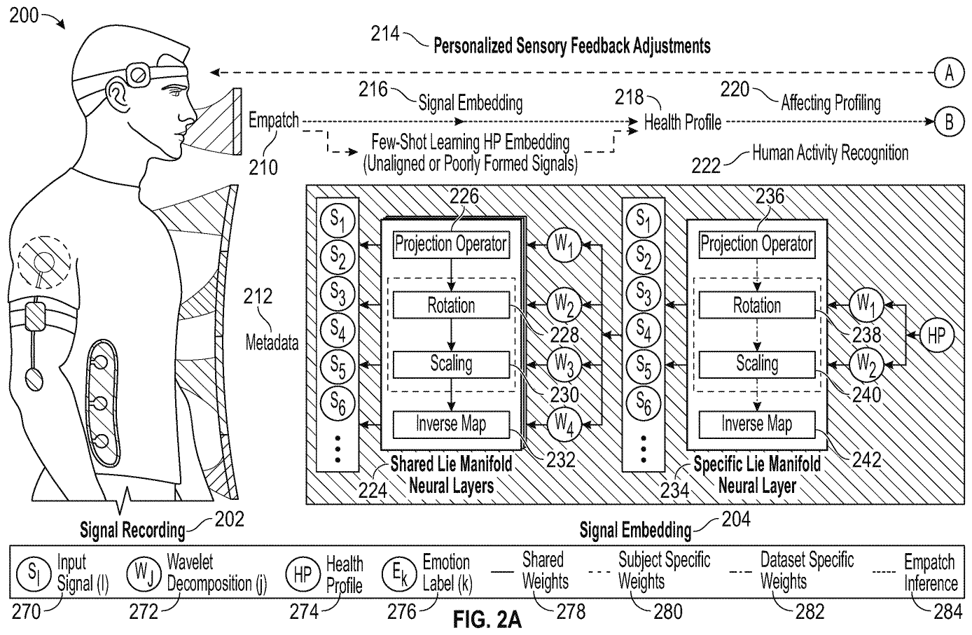

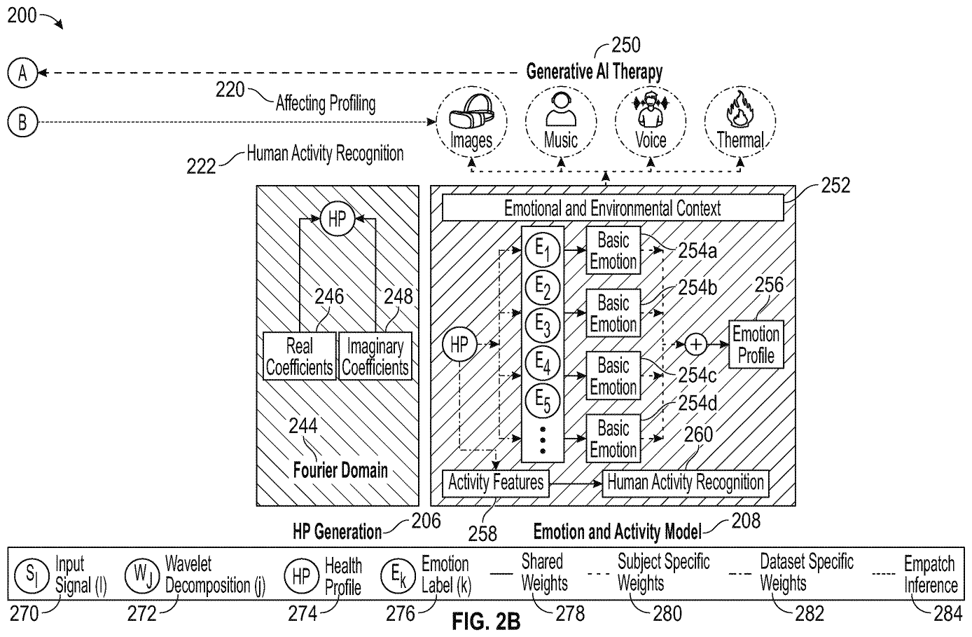

FIGS. 2A-B are diagrams depicting exemplary Lie manifold neural architectures for biomarker reconstruction and health profile generation, in accordance with various embodiments of the presently disclosed technology. The architectures shown generally at 200 illustrate how multimodal physiological time series can be transformed into a unified latent representation that supports reconstruction of physiological biomarkers, health profile formation, and downstream inference and intervention. Together, these figures depict both the structural components of the Lie manifold architecture and the signal processing pathway through which fragmented, irregularly sampled, and temporally misaligned physiological observations may be consolidated into a coherent, continuously updateable subject profile that supports interpretable, generalizable, and bidirectionally invertible affective intelligence.

In the example workflow, signal recording 202 may obtain physiological measurements from one or more sources, including, for example, an EMPATCH 210 wearable platform of the type described in connection with FIG. 1B (or any other physiological signal producing platform). Physiological signals acquired at this stage may span multiple sensing modalities, body locations, and devices that differ in sampling rate, data format, and temporal reference. As a practical matter, real-world wearable datasets frequently exhibit the kind of fragmentation and misalignment that makes conventional inference pipelines brittle. For example, EOG blink features may be culled during periods when a subject closes their eyes in response to a stressor, while EEG sensors on the same device may continue recording at a predetermined sampling frequency. The resulting data streams thus may have different temporal densities and different amounts of missing content even within a single session, let alone across sessions or across devices.

The recorded signals may be accompanied by metadata 212, which can include information such as modality identifiers, timestamps, device identifiers, context labels, signal quality indicators, and any other annotation that characterizes when, where, and how a particular observation was acquired. This contextual information may enable the subsequent model stages to operate coherently across heterogeneous devices and datasets while still maintaining a principled representation of what was observed and at what time, supporting the model's ability to treat each incoming data point as a time-stamped, modality-specific observable constraint rather than as an entry in a fixed, synchronous input vector.

The recorded observations may feed into a first representation stage shown as signal embedding step 216, which transforms raw or preprocessed physiological signals into an internal representation suitable for the Lie manifold architecture. The signal embedding step may compress high-dimensional signals, normalize representations across modalities and datasets to a common bounded range, and encode modality-specific temporal structure while preserving the information necessary to support later inversion and reconstruction. In practice, physiological signals from different datasets and sensing platforms may arrive with varying feature dimensionalities, representing different numbers of extracted biomarker features per modality, and the embedding step may produce a consistent internal representation regardless of these differences.

The embeddings produced at this stage can be understood as the model's interface between the raw observation space, where data arrives in heterogeneous form, and the structured geometric domain in which the Lie manifold transformations operate. Importantly, the embedding may be designed to be trainable in a reverse, decoder-oriented manner in which the model begins from a well-formed oversampled health profile representation and learns to generate, or reconstruct, the observable physiological signals from that profile. This reverse training strategy may allow the model to treat each observed signal timepoint as an independent constraint on the latent profile, discriminating improper profile embeddings by computing the reconstruction loss between the predicted hyper-sampled output and the actual recorded timepoints. The oversampled representation used as the training target may have a sampling frequency treated as a tunable hyperparameter set to exceed the Nyquist limit needed for fine-detail signal reconstruction, with the upper bound on temporal resolution already constrained by hardware-specific limitations, thereby achieving temporal alignment without introducing distortions to the ground truth signals.

The embedded representations may be processed through shared Lie manifold neural layers 224, which implement structured invertible transformations that are shared across tasks and, in various embodiments, across datasets and sensing platforms. The mathematical structure of these layers may be grounded in Lie group theory. At any given hidden layer, the linear component of the transformation may be expressed as a scaled Lie group element of the form A=a*exp(S), where S is a skew-symmetric matrix satisfying S=−S\hat{ }T, and the matrix exponential maps S from the Lie algebra so(n) into the Lie group SO(n). This mapping may induce an orthogonal linear rotation that preserves the signal's mean, variance, and L2-normalization, thereby maintaining biometric covariant properties and relative spatial relationships across hidden layers. The scalar a may act as a learnable normalization scale factor that provides controlled magnitude adjustments, modulating the Lipschitz constant of the transformation to stabilize intermediate signal amplitudes without requiring explicit spectral normalization strategies.

For signal embedding, human activity recognition, and affective profiling applications, the scale factor may be empirically bounded within a narrow range around unity, ensuring that the transformation does not induce large covariant shifts in the representations it processes. This volume-preserving and normalization-preserving character of the Lie group transformation may be in direct contrast to conventional unconstrained weight matrices, which can amplify, compress, or rotate representations arbitrarily, leading to vanishing or exploding gradients and making inversion difficult or numerically unstable.

Within the shared layers 224, the architecture applies these Lie group transformations not as standalone linear operations but within a learnable bijective nonlinear coordinate system, so that the overall transformation remains nonlinear with respect to the original reference frame, satisfying the universal approximation theorem requirement for at least one nonlinear operation. This may be achieved through a sequential structure of nonlinear compression, linear Lie group transformation, and nonlinear expansion. The nonlinear activation function used in this framework may be defined as a superposition of a linear term and a softsign function, parameterized by a linear coefficient that stabilizes gradients as the softsign converges, and a nonlinear coefficient that allows the function to dynamically adjust its linear and nonlinear regions. This construction ensures that the activation function maintains a non-zero first derivative everywhere, preventing vanishing gradients both at small and large signal amplitudes. If the model begins learning unphysically large amplitudes, the activation function collapses into an approximately linear transformation, preventing uncontrolled growth that would destabilize training. Conversely, by increasing the nonlinear dampening parameter in deeper layers, the model can modulate its effective complexity.

To prevent successive Lie group transformations from collapsing into a single composite operation, the architecture may alternate the nonlinear projections across successive layers, ensuring that each linear transformation remains distinct and is separated by a non-trivial nonlinear component. Within these shared layers, the architecture may include repeated compositions of a projection operator I 226, rotation I 228, scaling I 230, and an inverse map I 232. The projection operator maps the incoming signal into the structured subspace appropriate for the Lie group transformation, the rotation implements the orthogonal linear transformation parameterized by the matrix exponential of the skew-symmetric generator, the scaling applies the learnable normalization factor, and the inverse map provides the backward pathway that enables reconstruction and consistency enforcement. Because the Lie group transformation is inherently invertible, with the inverse corresponding to the transpose of the orthogonal rotation matrix or equivalently to the exponential of the negated generator, the inverse map can be computed without requiring auxiliary memory structures such as stored Jacobians, principal axes, or activation parameters that conventional reversible architectures typically require. This constant-time invertibility arises from the algebraic property that the inverse of a Lie group element exp(S) is exp(−S), which holds regardless of the values of the learned angular parameters across training updates. The result may be an architecture that is intrinsically and unconditionally reversible, meaning that inversion is not an approximation or a stored artifact but an algebraic consequence of the geometric structure of the transformations.

Alongside the shared transformations, the architecture can include at least one specific Lie manifold neural layer 234 tailored to a particular subspace, modality family, dataset, or downstream objective. This specific layer may be similarly expressed using a projection operator II 236, rotation II 238, scaling II 240, and an inverse map 242, with the same or similar fundamental mathematical structure as the shared layers but with parameters that are trained independently for each dataset or signal type rather than being shared across all inputs. The practical motivation for this division between shared and specific layers is that physiological signals from different platforms, body locations, and sensing modalities share common biometric and temporal structure reflecting the underlying physiology, while also exhibiting platform-specific characteristics related to electrode placement, sampling frequency, feature extraction methodology, and data collection protocol. The shared layers capture the generalizable physiological structure that is common across all datasets in the meta-training regime, while the specific layers adapt to the idiosyncrasies of each particular dataset or signal family.

In embodiments of the implemented architecture, the discrete wavelet transform decomposition within the specific layers may be stopped after one dyadic downsampling step, minimizing the number of dataset-specific parameters while still capturing signal-specific structure, whereas the shared layer decompositions continue until reaching a sequence length of 32 elements, chosen empirically to balance meaningful temporal information capture, lossless signal reconstruction, and training efficiency. This asymmetric depth allocation may ensure that the architecture remains parsimonious in its dataset-specific parameters while affording greater expressiveness to the shared components that generalize across platforms.

The Lie manifold processing can be performed in alternative representational domains. The figures illustrate a frequency-based representation through a Fourier domain 244, with real coefficients 246 and imaginary coefficients 248 depicted. Representing physiological signals in a frequency domain allows the architecture to learn transformations that operate on periodicity, spectral signatures, rhythmic oscillations, and other frequency-domain characteristics of physiological activity, such as the alpha band power of EEG signals or the spectral features of EDA, while maintaining the reversible geometric structure that enables reconstruction of time-domain signals when needed. The use of Fourier representations also provides a natural extension to complex-valued subspaces, wherein the skew-symmetric generator matrix is replaced by a skew-Hermitian matrix whose matrix exponential is a unitary transformation, extending the Lie group formalism from the real to the complex domain without disrupting the invertibility or the normalization-preserving properties of the architecture. However, the architecture may also incorporate biorthogonal wavelet neural operators that provide multi-resolution temporal analysis capable of capturing both long-term and transient signal patterns simultaneously.

Unlike Fourier-based methods, which rely on global sinusoidal basis functions that assume uniform sampling and can distort spectral representations when applied to irregularly sampled inputs, biorthogonal wavelet operators may provide localized time-frequency transformations that convolve along the signal and are therefore well-suited for capturing short-lived affective fluctuations and accommodating the temporal irregularities introduced by motion artifacts and data loss in real-world wearable datasets. The biorthogonal 3.1 wavelet family may be used because of its symmetry, lossless reconstruction property, and balanced time-frequency localization. Biorthogonal wavelets feature two dual wavelet pairs, one for decomposition and one for reconstruction, ensuring numerical consistency and lossless reconstruction across multiple hidden layers, which may be relevant for an invertible architecture that traverses the full model in both directions without accumulating numerical errors. The Lie manifold neural layers may be embedded within these wavelet neural operators, so that the architecture can apply structured geometric transformations jointly across both the approximate, or low-frequency, and detailed, or high-frequency, wavelet coefficient subspaces, learning both long-term temporal attention patterns and short-lived transient features while maintaining the geometric consistency and invertibility of the representation at each decomposition level.

Outputs of the manifold layers may be used for health profile generation 206, which may produce a unified latent health profile 218. The health profile may function as a continuously updateable, lossless snapshot of the subject's physiological and affective state, integrating fragmented, irregularly sampled, and temporally misaligned measurements from across all available modalities and datasets into a single coherent latent representation. Because the model is trained in reverse, learning to generate the health profile as the output that may account for all observable physiological signals by treating each recorded timepoint as an independent observable constraint, the resulting health profile carries biological significance in that its embedded dimensions correspond to interpretable physiological and temporal patterns rather than arbitrary latent codes.

The health profile sampling frequency may itself be a tunable hyperparameter that can be set to any desired temporal resolution, decoupled from the sampling frequencies of the input devices, providing a temporally unified representation that avoids the need for explicit resampling or interpolation of the raw signals. Because the mapping is invertible, the architecture may also enable biomarker reconstruction by applying the inverse mapping pathway to generate estimates of missing or unobserved physiological signal segments from the current state of the health profile. This reconstruction capability may be validated across missing data points spanning various biomarkers, demonstrating that the architecture can infer and regenerate absent signals from the concurrent physiological information embedded in the shared profile, thereby accommodating sensor dropout, data packet loss, and other forms of fragmentation without discarding any experimental instance that would otherwise fail a completeness requirement.

The generated health profile may drive multiple downstream inference heads that operate from the same or similar continuously updated latent representation. Affecting profiling 220 and human activity recognition 222 are illustrated as primary downstream tasks, and together they feed an emotion and activity model 208. The emotion pathway may perform a meta-analysis that extracts shared emotion primitives, referred to as basic emotions 254a-d, from the health profile, treating these primitives as building blocks for predicting complex emotional states with bidirectional traceability from model outputs back to the individual physiological observations that contributed to them.

Because the mapping is invertible, it is possible to trace which latent factors and which specific physiological constraints were most influential in producing a particular basic emotion prediction, providing interpretability that is not available in conventional forward-only black-box architectures. These basic emotion predictions may then be combined using user-specific weights to generate a personalized emotion profile 256 that accounts for inter-individual variability in how shared physiological patterns map to subjective affective experiences. Human activity recognition concurrently extracts activity features 258 and produces human activity recognition results 260 that provide contextual awareness for the affective inference, since the same physiological pattern, such as elevated EDA or altered EEG alpha power, may carry different affective significance depending on whether the subject is at rest, engaged in strenuous physical activity, or performing a demanding cognitive task.

The architecture may further support closed-loop use cases in which inferences inform interventions. The figures illustrate use of model outputs together with contextual inputs such as emotional and environmental context 252 to drive generative artificial intelligence therapy 250. In embodiments, this includes integration with large language model interfaces such as conversational artificial intelligence agents that receive the subject's current affective profile, including state anxiety scores derived from instruments such as the State-Trait Anxiety Inventory and positive and negative affect scores derived from instruments such as the Positive and Negative Affect Schedule, and generate personalized therapeutic responses tailored to the subject's current emotional state.

Image generation interfaces may additionally produce adaptive visualizations rendered in real time through virtual reality displays or holographic fan displays, aligning with subject-specific preferences to enhance engagement. The intervention pipeline may produce personalized sensory feedback adjustments 214, which represent adaptive control signals for auditory, thermal, visual, immersive, or other therapeutic outputs, thereby closing the loop from physiological sensing through latent profile construction to individualized real-time therapeutic response. The selection and parameterization of these interventions may be guided by an adaptive control policy that combines population-level affective priors encoded in a heuristic map with real-time subject-specific feedback accumulated as the health profile evolves, enabling the system to balance exploration of new intervention parameters against exploitation of parameters already known to be effective for the individual subject.

FIGS. 2A-B further depict an example multi-resolution signal processing pathway in which an input signal (i) 270 undergoes wavelet decomposition (j) 272 prior to health profile formation. The wavelet decomposition step may produce approximate, or low-frequency, coefficients that capture long-term temporal energy transfer and global temporal attention, as well as detailed, or high-frequency, coefficients that capture local transient structure including short-lived affective fluctuations and motion-related perturbations. Visualization of the rotational generator heatmaps within the detailed wavelet decomposition may reveal anti-diagonal banding patterns associated with long-term temporal attention, while the approximate coefficients may reveal phase-shifted, locally twisted structures corresponding to more compact, localized temporal features.

These internal geometric structures may be directly interpretable because the learned parameters of the Lie manifold architecture may correspond to physical quantities such as rotation angles and scaling factors, with all weights carrying explicit geometric meaning within a bounded, periodic learning domain. This interpretability may be a relevant property of the architecture that distinguishes it from conventional deep learning models whose internal representations lack an accessible geometric interpretation. From this multi-resolution processing, the system may output a latent health profile 274 that is used for downstream prediction, including generation of an emotion label (k) 276, while fully preserving the ability to map bidirectionally between the latent representation and the observation space through the invertible manifold transformations. The 3D evolution of the health profile across hidden layers and its 2D projection may illustrate how the signal flow organizes into structured geometric patterns that encode the temporal and cross-modal information content of the physiological observations in a differentiable, traversable latent space.

FIGS. 2A-B also illustrate a weight-sharing and adaptation strategy that enables the architecture to operate as a foundational model across diverse populations, datasets, and sensing configurations. The architecture may assign shared weights 278 to parameters that capture generalizable physiological structure applicable across all training datasets, subject-specific weights 280 to parameters that personalize the mapping for an individual subject's physiological characteristics and response patterns, and dataset-specific weights 282 to parameters that account for differences in sensor suites, sampling protocols, body locations, and collection environments between different data sources. This three-level weight hierarchy may allow a single trained model to generalize broadly while retaining the flexibility to adapt to new subjects and new platforms without requiring full retraining.

The progressive angular pruning mechanism inherent to the Lie manifold architecture may contribute to this capability by identifying and removing transformations whose learned angular parameters fall below a threshold, effectively collapsing those transformations to identity operations, since the exponential of the zero matrix is the identity matrix. Through this principled pruning process, the architecture may reduce its dataset-specific parameter complexity from an initial quadratic count proportional to the square of the signal dimension to a final approximately linear count, shifting the burden of representation from model parameters to the embedded health profile itself. This data-driven compression toward a dataset-specific compressibility limit may improve generalization on small datasets, avoid overfitting without imposing a priori sparsity constraints, and allow the effective model complexity to be determined adaptively from the data rather than by manual architecture design choices. The figure further depicts an EMPATCH inference 284 pathway to emphasize that the wearable platform can participate directly in real-time inference using the trained model, while the same model structure remains compatible with execution on a local embedded controller, a mobile device, or a remote compute resource, reflecting the flexible compute placement enabled by the lightweight and structured nature of the Lie manifold architecture.

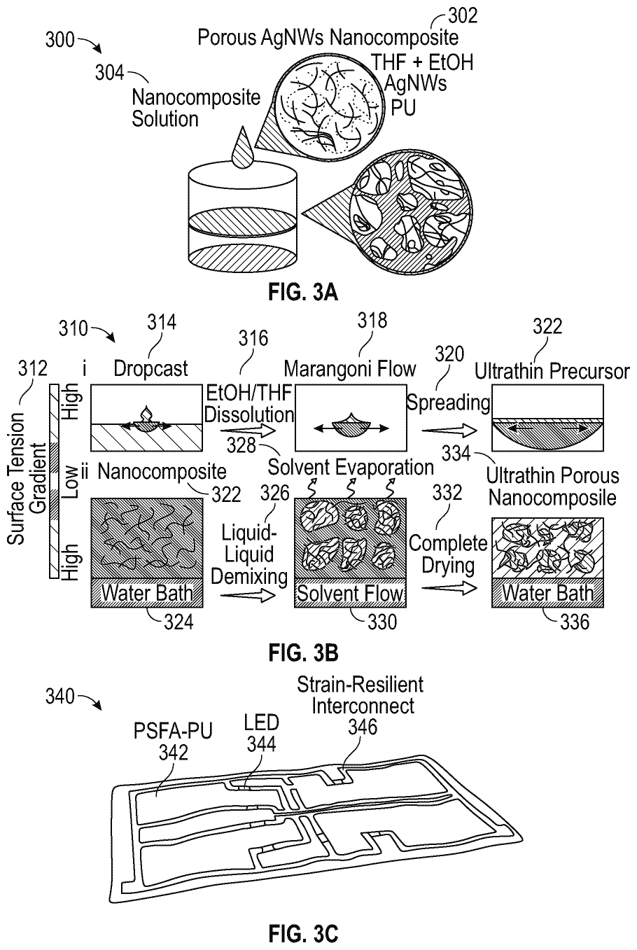

FIGS. 3A-B are diagrams depicting an example schematic of a phase-separated float assembly (PSFA) technique, in accordance with various embodiments of the presently disclosed technology. FIG. 3C is a diagram depicting an example schematic of light-emitting diode (LED) arrays fabricated with strain resilient interconnects, in accordance with various embodiments of the presently disclosed technology. Together, these figures illustrate the fabrication methodology underlying a wearable monitoring platform, detailing how ultrathin, elastic, breathable, and sweat-permeable nanocomposite electronic skins may be formed and how strain-resilient electrical interconnects are integrated to enable reliable optoelectronic and sensing functionality under repeated mechanical deformation. The PSFA process described in these figures addresses exemplary limitations of conventional wearable electronics fabrication, including reliance on time-intensive, cleanroom-dependent processes that constrain scalability and adaptability in large-area designs, by enabling rapid, ambient-condition formation of porous conductive films with properties specifically optimized for long-term conformal skin contact during high-motion and high-perspiration activities.

FIG. 3A, shown generally at 300, illustrates the precursor materials that may be used to form porous and mechanically compliant conductive layers for wearable bioelectronics. A porous silver nanowire (AgNW) nanocomposite 302, referred to herein as PSFA-PN, may be prepared from a nanocomposite solution 304 that combines polyurethane (PU) dissolved in tetrahydrofuran (THF) with silver nanowires dispersed in ethanol. In an example formulation, one milliliter of PU solution at a concentration of 50 milligrams per milliliter in THF is combined with 0.3 milliliters of AgNW solution at a concentration of 20 milligrams per milliliter in ethanol along with 0.1 milliliter of 1-butanol, which may serve to tune the phase-separation dynamics during film formation. The silver nanowires in this formulation carry amphiphilic surface ligands that govern their affinity for different phases of the polymer-solvent system and that ultimately drive their localization into the percolating conductive network.

The resulting nanocomposite may be tailored to provide a combination of electrical conductivity, stretch tolerance, and breathability that is well-suited for skin-compatible devices. Electrical conductivity may be further optimized through post-fabrication cold welding of the silver nanowire network using sodium borohydride, providing conductive performance comparable to metal-based electrodes while retaining the mechanical flexibility of the polymer matrix. The porous polyurethane substrate layer, referred to herein as PSFA-PU, may be fabricated using the same or a similar general process with a PU precursor in THF, and serve as a mechanically compliant host structure into which the PSFA-PN electrodes and strain-resilient interconnects are assembled.

FIG. 3B, shown generally at 310, depicts an example PSFA process sequence that transforms the nanocomposite solution into an ultrathin porous film through a sequence of interfacial and thermodynamic phenomena driven by solvent-water interactions at ambient conditions without requiring specialized cleanroom equipment or high-temperature processing. The process may begin with a dropcast 314 step in which the precursor solution is deposited onto the center of a water bath. Upon contact with the water surface, the THF and ethanol solvents in the precursor solution may begin to dissolve into the aqueous phase, locally reducing surface tension at the point of deposition. This reduction in local surface tension relative to the surrounding water surface establishes a surface tension gradient 312 between the deposition zone and the surrounding water surface, and the resulting thermodynamic imbalance drives Marangoni flow 318, a convective phenomenon in which fluid flows spontaneously from regions of low surface tension toward regions of higher surface tension. The Marangoni flow drives rapid lateral spreading 320 of the droplet boundary across the water surface, expanding the deposited material into an ultrathin precursor film 322 at the water-air interface over a timescale of seconds and without the need for mechanical spreading or substrate spinning. This spontaneous self-spreading behavior is relevant to the scalability of the PSFA process, as it enables formation of continuous ultrathin films over areas exceeding 200 square centimeters from a single dropcast event.

FIG. 3B further illustrates how the precursor film may be converted into a functional porous nanocomposite through a concurrent and subsequent sequence of phase-separation and drying events. Simultaneously with the Marangoni-flow-driven spreading, evaporation-driven liquid-liquid demixing 326 occurs within the nanocomposite film as the THF and ethanol solvents evaporate from the film surface and dissolve into the water bath below. As solvent is removed, the system may undergo spinodal decomposition, transitioning through a co-continuous phase formation regime in which polyurethane-rich and polyurethane-poor domains simultaneously develop and interconnect throughout the film volume. Due to the amphiphilic surface ligands on the silver nanowires and their immiscibility with the polyurethane polymer, the nanowires may preferentially partition into and localize within the polyurethane-poor domains as phase separation progresses. The path trajectory of the precursor solution through the phase diagram during this process transitions from co-continuous phase formation to spontaneous drying, yielding a continuous porous polyurethane structure in which the silver nanowires are confined to form interconnected percolation pathways through the pore-defining polyurethane-poor regions. As complete drying 332 is approached, ongoing solvent evaporation 328 and associated solvent flow 330 within the film continue to drive pore formation and consolidation, creating the interconnected pore network that defines the breathable, sweat-permeable architecture of the final film. A subsequent water bath 336 step may be employed for rinsing, stabilization, or additional phase-separation control to adjust the final porosity and mechanical characteristics of the film for specific applications. The film may then be left to dry before being collected using an acrylic frame for integration into the device assembly.

FIG. 3C, shown generally at 340, illustrates how the PSFA-derived substrate and porous nanocomposite materials may be incorporated into stretchable optoelectronic assemblies to demonstrate the breadth of functional electronic systems that can be realized using the PSFA fabrication platform. The PSFA-PU substrate 342 may serve as the mechanically compliant host matrix into which discrete electronic components are embedded and electrically interconnected. One or more LED elements 344 may be electrically coupled through strain-resilient interconnects 346 that are formed from a porous liquid metal composite fabricated following established methods for porous liquid metal composite preparation. The porous liquid metal composite used for these interconnects may combine the intrinsic fluidic deformability of liquid metal with the porous geometric architecture that enables stress redistribution during stretching, bending, and twisting.

As a result, the interconnects may maintain consistent electrical performance and uninterrupted circuit continuity during extensive mechanical deformation, including uniaxial stretching, biaxial stretching, bending, and twisting, as demonstrated by sustained illumination of the LED array throughout these deformation modes. The interconnects may also exhibit exceptional robustness against localized mechanical insults, having been shown to survive scalpel punctures, hammer impacts, and tweezer pricks without degradation of electrical performance. Sensor and interconnect patterns may be defined by laser cutting using a laser cutter prior to assembly, and the wearable system may be assembled by transferring predesigned PSFA-PN electrodes and strain-resilient interconnects onto the ultrathin PSFA-PU substrate using water-soluble tape as a transfer medium, with a waterborne polyurethane adhesive applied at all interfaces to ensure robust adhesion between the substrate, interconnects, and electrodes under strain. Real-time electrophysiological signal recordings, including electrocardiogram signals, acquired through the strain-invariant electrical wiring before and after stretching have confirmed that the porous liquid metal composite interconnect architecture may enable high-quality biopotential recording with negligible signal variation across mechanical deformation states, validating the suitability of this possible interconnect design for integration into wearable physiological monitoring devices that are subjected to continuous dynamic mechanical loading during normal daily use.

Collectively, FIGS. 3A-C illustrate a coherent and scalable fabrication approach for forming ultrathin, porous, mechanically compliant conductive nanocomposites and for integrating them with strain-resilient interconnect structures into fully functional soft electronic systems. The PSFA technique enables ambient-condition, large-area, single-step formation of breathable, sweat-permeable, skin-conformal electrode and substrate materials that outperform state-of-the-art alternatives fabricated using spin-coating and conventional float-assembly techniques across metrics including processing efficiency, large-area fabrication capability, breathability, wearability, sweat resistance, and electrical conductivity. These fabrication capabilities are relevant to various wearable monitoring platforms' ability to serve as a reliable, long-duration physiological data acquisition layer for the disclosed system, feeding high-fidelity multimodal physiological measurements into the Lie manifold-based health profile generation and downstream affective inference components described in connection with other figures.

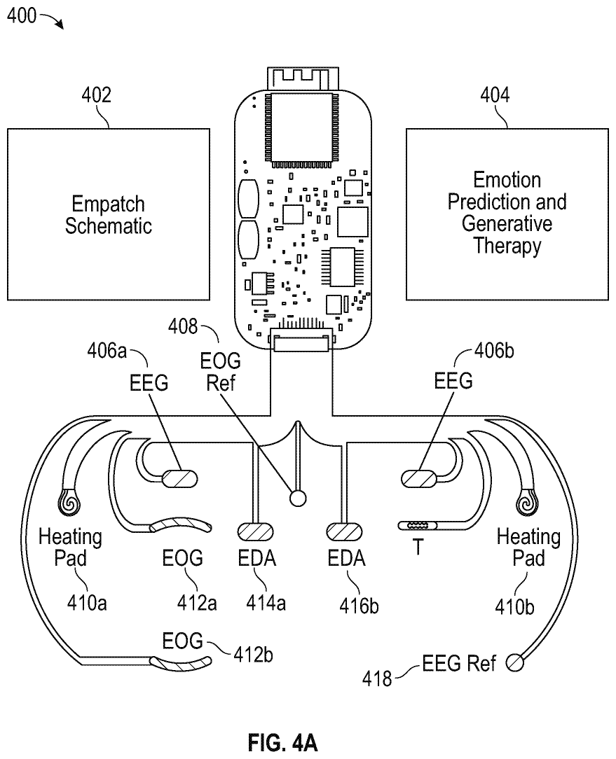

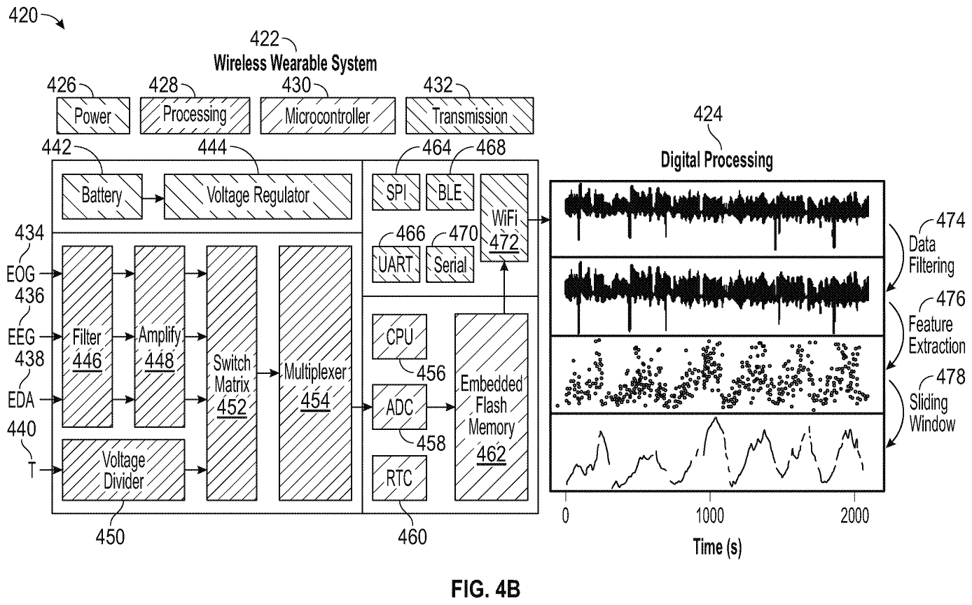

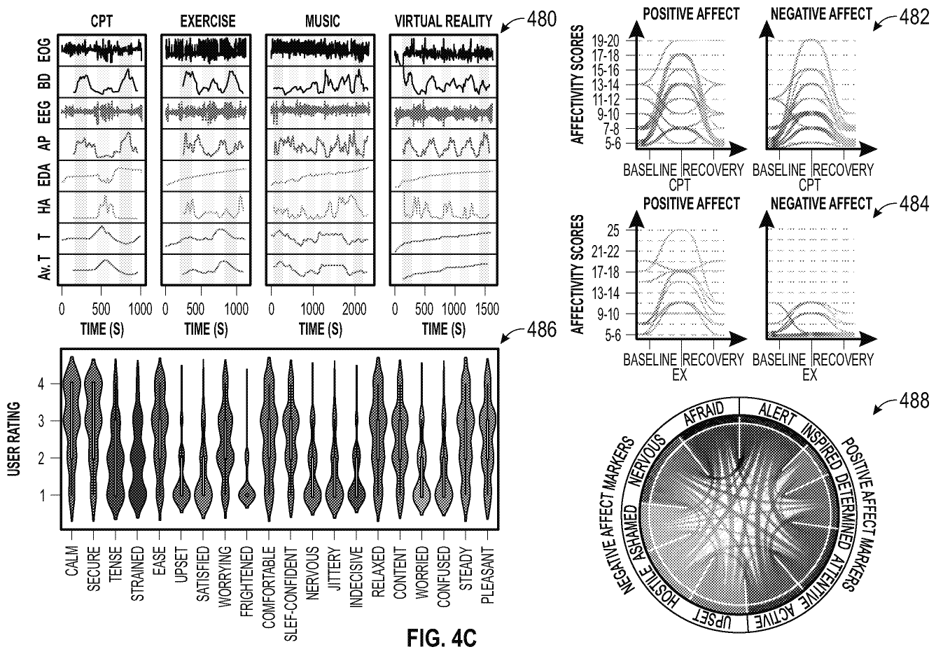

FIG. 4A is a diagram depicting an example schematic for a wearable patch platform for monitoring biomarkers and providing heat therapy, in accordance with various embodiments of the presently disclosed technology. The exemplary wearable monitoring system 400 depicted in this figure illustrates one representative hardware configuration through which multimodal physiological data may be acquired from a subject and through which sensory stimuli may be delivered in a closed-loop manner based on the outputs of the trained model and downstream emotion inference components described throughout this disclosure. The schematic 402 shows the spatial arrangement of sensing and stimulation elements as they may be positioned on the device, illustrating how the various physiological modalities are collected simultaneously from a single conformal on-body interface, and how the same platform that gathers the raw physiological inputs for health profile generation may also be capable of delivering therapeutic stimulation in response to the emotional state determinations made by the system. It should be understood that this hardware configuration is exemplary, and that one of ordinary skill in the art will appreciate that the disclosed trained model, invertible mapping, subject profile generation, and downstream inference and intervention capabilities may be implemented with a wide variety of physiological sensing platforms, body locations, electrode configurations, and stimulus delivery modalities, as discussed in further detail below.

The schematic 402 depicts the physical layout of sensing and stimulation elements which could be positioned on the forehead and periocular region of a subject's face, a body location that provides simultaneous access to cortical electrical signals, ocular movement potentials, electrodermal activity, and surface temperature from a single contiguous device footprint. The two EEG electrodes 406a and 406b may be positioned on the left and right sides of the forehead, respectively, following the FP1 and FP2 electrode positions of the standard 10-20 international electrode placement system, which may be established frontal scalp locations known to provide sensitivity to cortical activity associated with emotional processing, including frontal EEG asymmetry patterns that have been associated with valence-related affective states and with autonomic and central nervous system responses to emotionally significant stimuli. The EEG reference electrode 418 may be positioned on a neutral site, such as the side of the cheek or bony prominence behind the ear, to provide a stable electrical reference potential against which the cortical signals at FP1 and FP2 are differentially measured, thereby minimizing common-mode noise and electrode-to-electrode interference in the biopotential recordings. Prior to device application, the skin at each electrode site may be cleansed to optimize electrode-skin contact and reduce contact impedance, supporting stable biopotential acquisition throughout the monitoring session.

The EOG electrodes 412a and 412b may be positioned around the periocular region, with one electrode placed above the eye, or at the top position, and one electrode placed below the eye, or at the bottom position, to capture the vertical component of the corneo-retinal potential associated with vertical eye movements and blinks. The EOG reference electrode 408 may be positioned at a neutral site on the forehead, such as at the center of the forehead between the two EEG electrode locations, to provide the differential reference against which the periocular potential changes are measured. This differential configuration may allow the system to isolate ocular movement and blink-related signals from the background electroencephalographic activity, since changes in the corneo-retinal potential are directional and produce characteristic polarity patterns relative to the reference that are distinguishable from EEG signals at the same electrode sites. EOG signals acquired in this configuration may capture saccadic eye movements, fixation durations, blink rates, and blink durations, each of which can serve as a complementary indicator of cognitive load, arousal, and affective state that can be incorporated as an independent observable constraint on the subject's health profile by the domain-agnostic encoding and Lie manifold processing components of the system.

The EDA electrodes 414a and 416b may be positioned in adjacent locations on the forehead or periocular region, taking advantage of the measurable electrodermal response that can be recorded from the forehead and face in addition to more conventional palmar or plantar sites. EDA reflects changes in skin conductance driven by eccrine sweat gland activity under sympathetic nervous system control and provides a continuous, real-time indicator of autonomic emotional arousal. Temperature sensing, which may be accomplished through integrated thermal elements at the lateral regions of the head, may provide an additional channel of physiological information reflecting thermoregulatory responses that change with emotional state, physical exertion, and autonomic nervous system activation. Each of these sensing modalities may generate an independent time-series physiological measurement that can be transmitted to the wireless processing system for health profile embedding, and each may be present or absent at any given moment without disrupting the system's ability to maintain and update a coherent subject profile through the invertible mapping.

The heating pads 410a and 410b may be positioned at the left and right temple regions of the forehead, respectively, and may serve as the on-body thermal stimulation elements through which heat therapy can be delivered directly to the subject's skin in response to the emotional state determinations made by the downstream inference components of the system. Each heating pad may be fabricated on a copper-polyimide substrate via laser ablation and controlled through a microcontroller platform that regulates surface temperature within a therapeutic range, for example between approximately 35 and 50 degrees Celsius, calibrated through infrared camera measurements to ensure accurate temperature delivery within comfort and safety limits. The co-location of thermal stimulation and physiological sensing within the same conformal wearable platform may be a relevant feature of the closed-loop design, as it can allow thermal intervention to be delivered to the same body site from which physiological signals are concurrently being recorded, enabling the system to detect in real time the thermoregulatory and autonomic physiological changes that result from the thermal stimulus and to incorporate those changes into the evolving health profile for continued adaptive therapy selection.

The emotion prediction and generative therapy component 404 depicted in the figure may represent the software and algorithmic layer of the system that receives the multimodal physiological signals acquired by the sensing platform, processes them through the trained Lie manifold model to generate and update the subject health profile, performs downstream inference to determine the subject's current emotional state, and outputs control signals that govern the delivery of personalized sensory interventions. This component may encompass the full closed-loop pipeline from physiological data ingestion through health profile construction, affective state determination, and therapy selection and delivery, and may interface with the sensing and stimulation hardware shown in schematic 402 through the wireless wearable system described in connection with FIG. 4B. Together, the schematic 402 and the emotion prediction and generative therapy component 404 illustrate the complete end-to-end architecture of the exemplary wearable monitoring system 400 as an integrated sensing, inference, and intervention platform. As emphasized above, the particular sensor types, electrode positions, stimulation modalities, and hardware configuration shown in this figure represent one concrete and validated implementation of the disclosed system and should not be construed as limiting the scope to any particular sensing modality, body location, electrode material, or hardware form factor.

FIG. 4B is a block diagram depicting an example wireless wearable system and digital processing pipeline that can interface with the wearable monitoring platform, in accordance with various embodiments of the presently disclosed technology, shown generally at 420. The illustrated arrangement may include a wireless wearable system 422 that acquires multimodal physiological signals and performs local signal conditioning and communications, and a digital processing pipeline 424 that converts acquired physiological data into representations suitable for downstream health profile generation, affective inference, and closed-loop control. Together, these two components may form the hardware and signal processing substrate through which raw physiological measurements can be transformed into the standardized inputs consumed by the trained Lie manifold model described in connection with FIGS. 2A-B. It should be understood that the particular hardware configuration illustrated here is one exemplary implementation, and that the trained model, invertible mapping, and subject profile generation components of the disclosed system are compatible with any hardware and signal processing arrangement capable of producing multimodal physiological time-series data in a form that can be passed to the domain-agnostic encoding stage.

The wireless wearable system 422 may receive physiological inputs from sensing channels that may include EOG 434, EEG 436, EDA 438, and skin temperature 440. Each of these channels may reflect a distinct and complementary dimension of the subject's physiological and affective state, and together they may constitute the multimodal observable input to the health profile generation system. Because the domain-agnostic encoding component of the trained model may be designed to accommodate any available subset of these modalities at any given time, the wireless wearable system 422 need not guarantee simultaneous availability of all channels, and the system may remain fully (or partially) functional when one or more sensing channels are intermittently absent, degraded, or operating at reduced sampling frequency.

The physiological signals received from these channels may be routed to an analog and mixed-signal front end that performs conditioning prior to digitization. A filter stage 446 may apply band-limiting and artifact attenuation to remove out-of-band noise, powerline interference, and motion artifacts that would otherwise corrupt the digitized physiological signals. An amplification stage 448 may apply appropriate gain and dynamic range matching to bring the conditioned signals within the input range of the analog-to-digital converter, accounting for the low-amplitude nature of biopotential signals such as EEG and EOG, which typically fall in the microvolt to millivolt range and require careful gain staging to avoid clipping or loss of resolution. A voltage divider 450 may provide scaling or biasing of signals where appropriate, for example to center signal waveforms within the converter's input common-mode range or to accommodate differences in signal amplitude across sensing modalities. The conditioned signals may be routed through a switch matrix 452 and a multiplexer 454 that together support flexible channel selection, reconfiguration of electrode pairings for differential or referential measurement configurations, and time-multiplexed acquisition that can allow a single analog-to-digital conversion chain to serve multiple sensing channels efficiently, reducing hardware complexity and power consumption without sacrificing the temporal resolution required for real-time physiological monitoring.

The wireless wearable system 422 may further include core embedded electronics that coordinate acquisition timing, digitization, local computation, and data management. A central processing unit 456 may orchestrate the sampling sequence, manage buffering of digitized samples, coordinate communication with peripheral modules, and may perform lightweight on-device computation such as feature extraction or model inference when sufficient local compute resources are available. An analog-to-digital converter 458 may digitize one or more conditioned analog channels at the sampling rates appropriate for each physiological modality, which may differ across modalities reflecting the different frequency content of EEG, EOG, EDA, and temperature signals. A real-time clock 460 may provide accurate timekeeping to support consistent timestamping of acquired samples and coordination across simultaneously operating sensing channels, which may be relevant for maintaining temporal metadata that allows the trained model to incorporate each incoming measurement as a time-stamped observable constraint on the subject profile. Embedded flash memory 462 may provide local storage for digitized samples, extracted features, calibration parameters, model parameters, and event logs, enabling the system to continue buffering physiological data and supporting local inference even when a wireless communications link is intermittent or temporarily unavailable. Internal communication between modules, sensors, converters, and radios is supported through serial peripheral interface 464 and universal asynchronous receiver-transmitter 466 interconnects, which may provide standard, low-overhead digital communication pathways appropriate for the data rates and latency requirements of wearable physiological monitoring systems.

The wireless wearable system 422 may also include a power and communications subsystem designed to support untethered, continuous operation throughout extended monitoring sessions. A battery 442 may provide onboard energy storage, and a voltage regulator 444 may generate stable regulated supply rails for both the analog front-end circuitry, which may require low-noise power to avoid introducing artifacts into sensitive biopotential measurements, and the digital processing and communications blocks. The functional roles across the wearable platform may be partitioned into power management 426, signal processing 428, microcontroller coordination 430, and wireless transmission 432, reflecting a modular architecture that allows each subsystem to be independently optimized for power efficiency, performance, and reliability.

For wireless communications, the transmission subsystem 432 may support one or more communication links including Bluetooth Low Energy 468, which can enable low-power streaming of physiological data and metadata to a companion mobile device or local compute resource; serial communication 470, which may support wired data transfer for configuration, debugging, or high-bandwidth streaming applications; and WiFi 472, which may enable higher-throughput data transfer to a remote compute resource or cloud processing environment when bandwidth or latency requirements exceed what is achievable over a Bluetooth link. These communication options may collectively allow the system to stream physiological data and associated metadata to an external computing device in real time, as well as to receive configuration updates, calibration commands, model parameter updates, and closed-loop therapy control signals from the downstream processing and inference components of the system.

The digital processing pipeline 424 may be executed on the wearable device itself, on a companion mobile device, or on a remote compute resource that receives streamed data from the wireless wearable system 422, reflecting the flexible compute placement enabled by the modular architecture of the overall system. Regardless of where computation is performed, the digital processing pipeline 424 may apply a consistent sequence of operations to transform the raw digitized physiological signals into standardized representations suitable for input to the trained model. A data filtering stage 474 may apply additional digital domain processing to further attenuate noise, remove residual motion artifacts, suppress baseline wander, and enforce band-limiting constraints appropriate for each physiological modality, complementing the analog-domain conditioning performed in the wireless wearable system 422 and ensuring that the representations entering the feature extraction stage reflect the true underlying physiology rather than acquisition artifacts. A feature extraction stage 476 may compute signal representations that capture the physiologically and affectively relevant information content of each sensing channel, including spectral band powers, time-frequency features, statistical descriptors, and other domain-specific representations that have established relationships to the physiological and affective quantities of interest.

For EEG signals, this may include computation of power spectral density estimates in the delta, theta, alpha, beta, and gamma frequency bands, as well as frontal asymmetry indices derived from differential band power measures at FP1 and FP2. For EDA signals, this may include separation of the tonic and phasic components of the conductance signal, extraction of skin conductance response amplitude, latency, and recovery time features, and computation of the overall conductance level. For EOG signals, this may include detection and characterization of blink events and saccadic eye movements. For temperature signals, this may include computation of mean, variance, and rate-of-change features capturing the temporal dynamics of thermoregulatory responses.

A sliding window mechanism 478 may support continuous, real-time processing of these features over overlapping temporal windows of configurable length and step size, enabling the trained model to receive a continuous stream of updated feature representations that can be incorporated as new observable constraints on the subject health profile without requiring the accumulation of a complete, fixed-length data epoch before each profile update. This windowed streaming architecture may be aligned with the incremental profile update capability of the invertible mapping described throughout this disclosure, in which each new measurement window is mapped into the latent space and used to refine the subject profile as new physiological information becomes available, supporting low-latency emotional state determination and real-time adaptive therapy control.

FIG. 4C is a diagram depicting various exemplary physiological responses and features across sensing platforms and/or conditions, in accordance with various embodiments of the presently disclosed technology. FIG. 4C illustrates how physiological measurements and derived features can vary across different experimental or real-world conditions, and how those variations may be expressed as signals, features, markers, and user-reported outputs that can serve as inputs to the health profile generation and downstream affective inference components of the present disclosure. The physiological response patterns and derived features depicted in this figure may represent examples of the kinds of observable constraints that the domain-agnostic encoding and invertible mapping components of the trained model may be designed to ingest, map into the latent subject profile, and use to support emotional state determination and adaptive therapy selection.

The conditions shown at 480 may include cold pressor test (CPT), exercise, music, and virtual reality, each of which may be capable of eliciting a distinct and recognizable pattern of physiological and affective responses that differs in its autonomic, cortical, and subjective characteristics. The cold pressor test may involve immersion of a limb in cold water to induce a controlled physiological stress response, producing well-characterized increases in sympathetic nervous system activation that are reflected in EDA, heart rate, and peripheral temperature signals. Exercise may elicit cardiovascular and thermoregulatory responses that produce characteristic changes across multiple physiological modalities and that must be disambiguated from stress-related arousal responses when the system is inferring affective state from concurrent physiological activity, a disambiguation that the human activity recognition component of the architecture is specifically designed to support. Music and virtual reality may represent stimulus-driven conditions in which sensory input can be used to modulate affective state, and the physiological signatures associated with these conditions can serve both as validation data for the system's emotion inference components and as reference patterns for assessing the effectiveness of auditory and immersive visual therapeutic interventions of the type described in connection with FIGS. 1A and 1C. By evaluating physiological feature behavior across this range of stress-inducing, activity-driven, and stimulus-driven contexts, the system can learn condition-sensitive representations within the shared Lie manifold layers that generalize across diverse real-world usage scenarios.

The positive affect and negative affect graphs shown at 482 for the cold pressor test condition and at 484 for the exercise condition may illustrate that affect-related outcomes can be associated with condition-specific physiological signatures and can be represented as time-varying trajectories that differ systematically across conditions. Positive affect and negative affect may be operationalized here as continuous scalar scores derived from affective rating instruments such as the Positive and Negative Affect Schedule, which can quantify the intensity of experienced positive and negative emotional states on independent dimensions rather than treating affect as a single bipolar axis. The temporal trajectories of these scores across the cold pressor test and exercise conditions may reflect the dynamic nature of affective experience during physiologically engaging tasks, capturing how affect evolves over the course of a condition rather than remaining static, and illustrating the importance of continuous physiological monitoring and incremental profile updating for tracking these moment-to-moment changes. In some implementations, positive and negative affect traces may be differentiated using different grayscale tones and line styles to support visual comparison when reproduced in monochrome.

The affect-related markers shown at 486 may represent discrete events, threshold crossings, peaks, or windows of interest derived from the physiological data and corresponding affective annotation labels, marking specific timepoints or intervals at which affective state transitions, peaks of arousal, or significant changes in the positive or negative affect trajectory occur. These markers may be illustrative of the kind of temporally localized affective annotations that can serve as training targets or evaluation reference points for the downstream inference components of the system, and they may reflect the temporal granularity at which the health profile can be expected to capture and reflect changes in the subject's affective state.

The user rating chart shown at 488 may represent subjective self-report feedback collected from the subject during or following a monitoring session, which may be used as ground truth supervision for training the affective inference components of the system, as validation data for assessing the correspondence between model-inferred emotional state estimates and the subject's own experience, and as a source of personalization signal that can be incorporated into the subject-specific weight adaptation mechanism described in connection with FIG. 2B. The combination of continuous physiological feature trajectories, time-localized affective markers, and subjective user ratings shown collectively across 480 through 488 may illustrate the multi-level, multi-resolution nature of the affective supervision signal available to the system, and demonstrates how physiological observables, derived features, and self-reported experience can be jointly used to train, validate, and personalize the health profile generation and downstream inference components of the disclosed architecture.

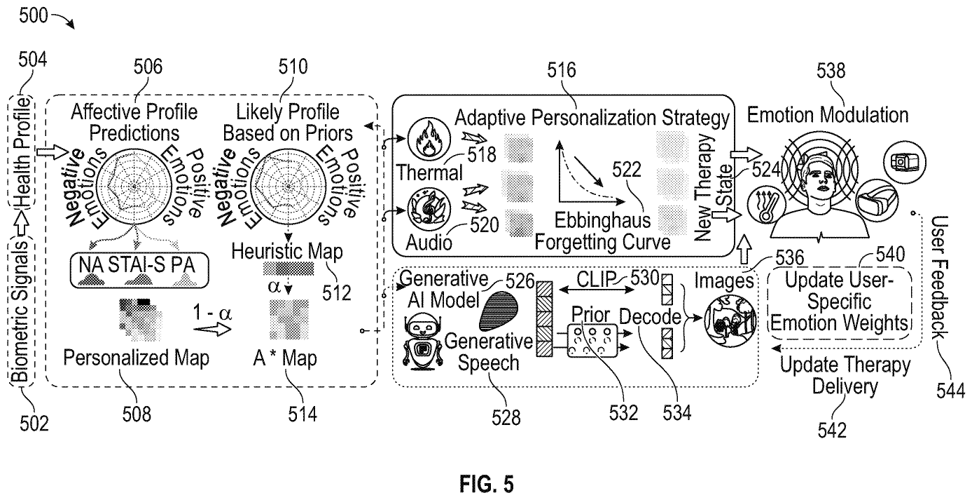

FIG. 5 is a diagram depicting an example schematic of an emotion modulation therapy algorithm, in accordance with various embodiments of the presently disclosed technology, shown generally at 500. The illustrated workflow may represent the closed-loop computational pipeline through which multimodal physiological observations may be transformed into personalized, adaptive therapeutic interventions, integrating physiological sensing, latent profile-based affective inference, heuristic and search-based therapy planning, generative content production, and continuous subject-specific personalization into a unified real-time decision and delivery system. Each stage of the workflow may be enabled by and grounded in the health profile generation and invertible mapping capabilities described in connection with FIGS. 2A-B, and the adaptive personalization and feedback mechanisms illustrated here may represent the closed-loop operational mode of the broader architecture described in connection with FIGS. 1A and 1C.

The workflow may begin with collection of biometric signals 502, which may include multimodal physiological measurements and derived features obtained from one or more sensing devices of the type described in connection with FIGS. 4A and 4B. These biometric signals may encompass a wide range of physiological modalities available from the sensing platform, including electroencephalographic signals, electrooculographic signals, electrodermal activity, and skin temperature, as well as any derived spectral, statistical, or time-frequency features extracted from those raw signals. The biometric signals 502 may be processed by the trained Lie manifold model to generate or update a health profile 504, which may serve as the continuously maintained unified latent representation of the subject that aggregates physiological observations across time and across modalities, treating each incoming measurement as an independent observable constraint that can refine the profile without requiring complete or synchronized input across all sensing channels. The health profile 504 may thus capture not only the subject's instantaneous physiological condition but also the temporal trajectory of that condition, encoding trends, dynamics, and cross-modal relationships that may be relevant to both accurate affective state inference and effective therapy selection.

The health profile 504 may be used to produce an affective profile prediction 506 that represents an estimated affective condition, temporal trend, or probability distribution over affective states for the subject at the current moment. The affective profile prediction 506 may be computed from the health profile 504 through the downstream inference heads of the Lie manifold architecture, or via one or more intermediate representations that extract task-specific features from the latent profile before applying affective classification or regression logic. In practice, the affective profile prediction may be expressed as a combination of continuous affect dimension scores, such as valence and arousal, alongside discrete emotion class probabilities and higher-level psychological state estimates such as state anxiety scores and positive and negative affect schedule measurements, providing a multi-resolution characterization of the subject's current affective condition that is richer than any single-dimensional output. The affective profile prediction 506 may provide the primary input to the therapy selection and delivery workflow, which may aim to shift the subject's affective condition from its current estimated state toward a desired target state through the adaptive delivery of sensory interventions.

The therapy selection workflow may incorporate a personalized map 508 that encodes how a particular subject may be expected to respond to the available therapy options given their current affective state, physiological profile, and prior response history. The personalized map 508 may be initialized using a likely profile based on priors 510, which can reflect population-level affective response patterns derived from the training datasets, previously observed individual response patterns accumulated over prior monitoring sessions with the same subject, or other baseline assumptions appropriate to the deployment context. This prior-based initialization may allow the system to make reasonable therapy selections from the first interaction with a new subject, before sufficient subject-specific response data has been accumulated to support fully individualized decision-making. The personalized map 508 may also incorporate a heuristic map 512 that encodes simplified decision rules or cost functions used to guide therapy selection, for example by associating specific ranges of affective profile prediction outputs with recommended intervention types or by imposing constraints on therapy sequencing, duration, or intensity that reflect clinical or safety considerations. Additionally, the personalized map 508 may incorporate a planning structure 514 that represents a search-based mechanism for selecting a sequence of therapy actions intended to move the subject from their current estimated affective state toward a target affective state over a planning horizon, accounting for the expected temporal dynamics of therapeutic response and the need to balance immediate intervention effects against longer-term affective trajectory objectives.

The therapy decision logic may be implemented through an adaptive personalization strategy 516 that selects therapy modalities and delivery parameters at each decision step and continuously updates those selections as new physiological observations refine the health profile and as observed affective responses provide feedback on the effectiveness of prior interventions. The interventions available to the adaptive personalization strategy may include thermal outputs 518, delivered through the on-body heating elements described in connection with FIG. 4A, and audio outputs 520, delivered through speakers, headphones, or other auditory transducers. These modalities may be delivered individually or in combination, with the adaptive strategy determining the optimal pairing, sequencing, and parameterization of modalities based on the personalized map and the current affective profile prediction. The adaptive personalization strategy may model the temporal dynamics of therapeutic habituation and effect decay using the Ebbinghaus forgetting curve 522, an empirical model of memory and learning retention that describes how the strength of an acquired response decays exponentially over time in the absence of reinforcement. By incorporating the forgetting curve into the therapy scheduling logic, the system can anticipate when the therapeutic effect of a previously delivered intervention is expected to have diminished below a target threshold and can proactively schedule reinforcement, adjust stimulus intensity, or transition to a different modality before the subject's affective state regresses, thereby maintaining sustained therapeutic benefit over extended monitoring sessions. Based on these inputs, the workflow may transition to a new therapy state 524 that represents an updated therapy plan and delivery configuration reflecting the combined output of the personalized map, heuristic rules, planning logic, and forgetting curve scheduling.

The workflow may also include generative content components implemented by a generative artificial intelligence model 526 that produces dynamically personalized therapeutic content in real time. The generative AI model 526 may produce generative speech 528 for audio-based therapeutic delivery, including personalized guided relaxation scripts, motivational coaching, mindfulness instructions, or affective cueing that can be adapted to the subject's current estimated emotional state and delivered through the audio output channel. For visual content generation and selection, the generative AI model 526 may employ a vision-language model such as CLIP 530 to support the selection, ranking, and semantic conditioning of visual therapeutic content based on the subject's affective profile and expressed or inferred preferences. Content generation and selection may be guided by a learned prior 532 that encodes distributional knowledge about which types of visual and auditory content are most effective for subjects with particular affective profiles, followed by a decode operation 534 that produces deliverable outputs including images 536 rendered through visual display systems such as virtual reality headsets or holographic fan displays. The generative content pipeline may thus enable the system to move beyond fixed, pre-authored therapeutic stimuli toward dynamically generated, semantically coherent, and affectively appropriate sensory experiences that can be continuously adapted to the evolving state of the individual subject.

The outputs of the therapy planning and generative content components may be applied to drive emotion modulation 538, representing the delivery of the selected therapeutic stimuli to the subject through the available output modalities. Following delivery, the system may update its internal state through two parallel adaptation mechanisms. The first, update user-specific emotion weights 540, may adjust the learned parameters that encode how a particular subject responds to different interventions and how their physiological signals map to affective states, incorporating the observed response to the most recently delivered intervention to progressively personalize the mapping between physiological observables and affective states for the individual subject. This weight update mechanism may correspond to the subject-specific weight adaptation layer described in connection with FIG. 2B, and it may allow the system to become increasingly accurate and effective over repeated monitoring sessions with the same subject. The second adaptation mechanism, update therapy delivery 542, may adjust the operational parameters of the therapy delivery system itself, including modality selection, stimulus timing, duration, intensity, and sequencing, based on the measured physiological response and the updated affective profile prediction, thereby refining the delivery configuration to better match the subject's current therapeutic needs.

The loop may be closed through user feedback 544, which may include explicit self-reports such as affect ratings or comfort assessments provided by the subject through a user interface, implicit behavioral signals such as changes in engagement or response latency, physiological response indicators automatically derived from the updated health profile following intervention delivery, or any combination of these feedback sources. User feedback 544 may be used to validate whether the anticipated affective change produced by the delivered intervention actually occurred, to recalibrate the personalized map 508 by updating the association between intervention parameters and observed affective outcomes, to refine both the prior based on subject history 510 and the generative content prior 532 with newly acquired evidence about the subject's preferences and responsiveness, and to update the current therapy state 524 to reflect the most recent evidence about what interventions are working and what adjustments are warranted. In this way, FIG. 5 illustrates an adaptive closed-loop emotion modulation algorithm in which physiological sensing, latent profile-based affective inference, heuristic and search-based planning, generative content production, and multi-channel personalization may be continuously integrated and mutually reinforcing, enabling the system to converge over time toward an intervention strategy that is tailored to the individual subject's physiology, affective response patterns, and therapeutic needs.

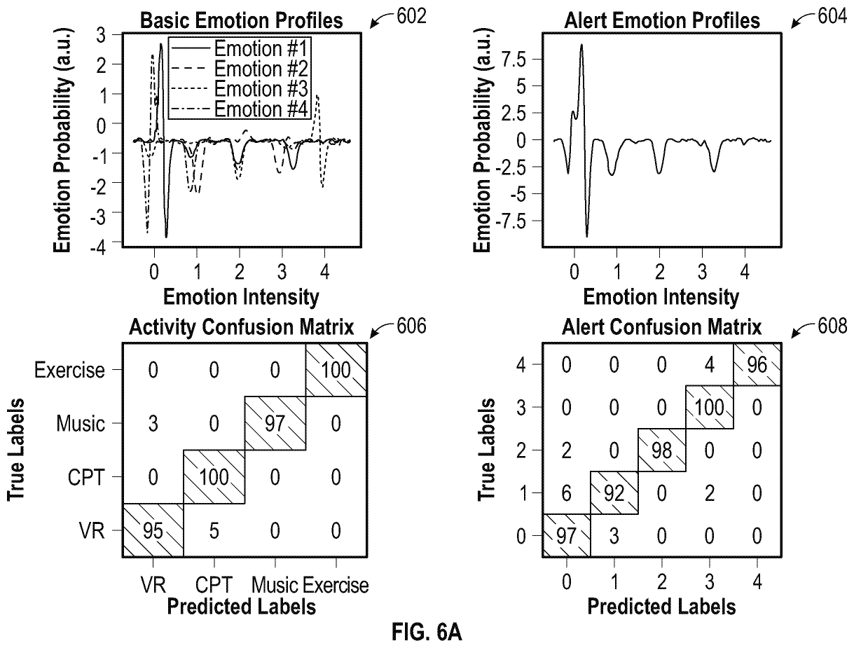

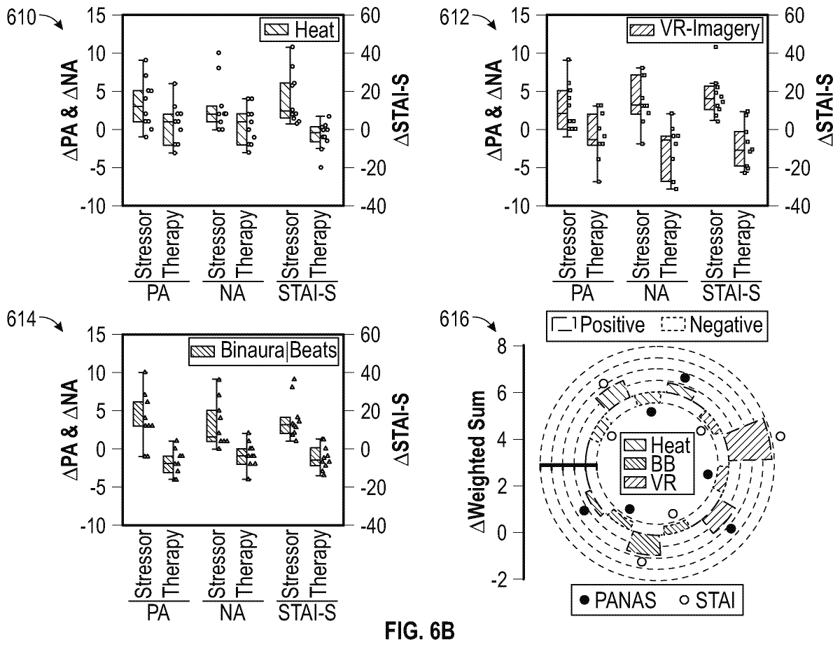

FIGS. 6A-B are diagrams depicting various exemplary health profile distributions, confusion matrices, therapy effectiveness comparisons, and emotion modulation effects, in accordance with various embodiments of the presently disclosed technology. Collectively, these figures illustrate example outputs that may be produced when multimodal physiological data is transformed into a unified subject profile using the Lie manifold architecture described herein and then used for downstream classification, therapy evaluation, and closed-loop assessment of intervention effects. The results shown in these figures may be derived from a health profile of at least 256 elements, and class intensities may be extracted from each output distribution by dividing the profile dimension into even class segments, with a final emotion probability distribution expressed as raw unnormalized logits, providing a concrete illustration of how the latent subject profile supports interpretable, quantitative affective inference across multiple downstream tasks simultaneously.

FIG. 6A includes basic emotion profiles 602, which may represent example distributions of inferred emotional representations derived from physiological observations that have been embedded into the latent health profile and then decoded through the downstream affective inference components of the architecture. Each basic emotion profile may correspond to one of the fundamental emotion primitives extracted from the health profile dimension, and the distribution of probability mass across that dimension may reflect the degree to which the observed physiological state is consistent with each emotion class. These profiles may illustrate that different emotional conditions, including the four basic emotion classes labeled as emotions one through four in the illustrated example, form distinct and separable groupings within the shared latent representation space, with each class occupying a characteristic region of the health profile dimension that may be reliably activated by the physiological signatures associated with that emotional condition. The degree of separability among the basic emotion profile distributions may be indicative of how well the learned subject profile supports emotion-related inference across subjects, sessions, and sensing configurations, and it may reflect the architecture's ability to extract shared emotion primitives that may be consistent and reproducible within the unified latent space despite the variability introduced by heterogeneous sensor inputs, fragmented data, and individual differences in physiological expression of emotional states.

FIG. 6A further includes an alert emotion profile 604, which may represent an example profile distribution associated with an alertness-related complex emotional state. Unlike the basic emotion profiles, which may correspond to fundamental affective primitives extracted from the health profile dimension, the alert emotion profile may represent a complex emotion class whose physiological correlates span multiple dimensions of the latent health profile and reflect the combination of autonomic activation, cortical engagement, and attentional orienting associated with an alert state. In the illustrated example, the alert emotion profile may exhibit a characteristic distribution shape centered at a different region of the health profile dimension than the basic emotion profiles, illustrating that the latent representation space may accommodate both fundamental and complex emotional states in a geometrically structured manner that supports their joint inference from the same underlying profile. In certain implementations, the alert emotion profile 604 can serve as a reference distribution for comparing state-dependent shifts in physiological representations, for quantifying transitions between affective conditions over time, and for assessing how consistently a given complex emotional state is represented across different subjects and data sources within the unified latent space.

FIG. 6A also includes an activity confusion matrix 606 and an alert confusion matrix 608. The activity confusion matrix 606 may be generated during evaluation of the human activity recognition component of the downstream inference pipeline and quantify the correspondence between predicted and true activity class labels across the four sensory and physical conditions evaluated in the dataset, namely virtual reality, cold pressor test, music, and exercise. The illustrated confusion matrix may demonstrate high classification accuracy across all four activity classes, with the majority of predictions falling on the diagonal, reflecting reliable separability of distinct physical and sensory conditions within the health profile-based representation. Specifically, the activity recognition results may show classification accuracies of approximately 100 percent for virtual reality, 100 percent for cold pressor test, 98 percent for music, and 97 percent for exercise, confirming that the unified latent health profile captures condition-specific physiological patterns with sufficient fidelity to support reliable activity disambiguation.

The alert confusion matrix 608 may be generated during evaluation of complex emotion classification within the same dataset, specifically assessing the system's ability to predict the alert emotional state from the health profile. The alert confusion matrix may demonstrate similarly high classification performance, with accuracy values of approximately 96 percent for virtual reality, 92 percent for cold pressor test, 98 percent for music, and 100 percent for exercise, indicating that the latent health profile supports reliable inference of complex emotional states as well as physical activity conditions. Together, the activity confusion matrix 606 and alert confusion matrix 608 may confirm that the profile-based inference pipeline generalizes across both activity recognition and emotion classification tasks simultaneously from the same shared latent representation, without requiring separate preprocessing pipelines or task-specific feature engineering for each downstream objective.

FIG. 6B includes emotion intensity measures 610 and 612, which may represent example intensity outputs computed across emotional conditions and evaluation windows for two separate configurations of the downstream inference pipeline. Emotion intensity may be computed from the health profile output distribution by integrating the probability mass assigned to each class segment, providing a scalar measure that reflects the degree of confidence with which the system associates the current health profile state with a particular emotional condition. In the illustrated examples, the emotion intensity measures may be computed across multiple conditions spanning both a stressor phase, corresponding to the cold pressor test, and a therapy phase, during which one of the three available therapeutic intervention modalities may be delivered, enabling comparison of inferred emotional condition intensity before and after intervention. The two emotion intensity panels 610 and 612 may reflect different parameterizations of the downstream inference logic, different subject-specific weight configurations, or different intervention modalities being evaluated, thereby illustrating the range of intensity trajectories that may be observed across subjects and conditions and providing a basis for assessing the sensitivity of the inference pipeline to changes in the subject's affective state induced by therapy delivery.

FIG. 6B further includes predicted labels 614 and 616, which may represent the discrete class label outputs produced by the downstream affective inference heads at each evaluation window, corresponding respectively to the two emotion intensity configurations depicted in 610 and 612. The predicted labels panels may illustrate the temporal sequence of emotion class assignments produced by the system across the stressor and therapy phases of the evaluation protocol, enabling visual inspection of how the inferred emotional state transitions in response to the cold pressor test stressor and subsequently to the delivered therapeutic intervention. In the illustrated examples, the predicted labels may show systematic shifts in the dominant emotion class assignment between the stressor and therapy phases, consistent with the physiological and affective changes that would be expected when a stressor-induced negative affective state is modulated by an appropriate therapeutic intervention. Together, the emotion intensity measures and predicted labels shown in 610 through 616 may provide complementary quantitative evidence of the system's ability to detect meaningful changes in emotional state from the evolving health profile and to attribute those changes to the delivered intervention, thereby validating the closed-loop sensing, profiling, inference, and therapy delivery pipeline as a complete and functional emotion modulation system.

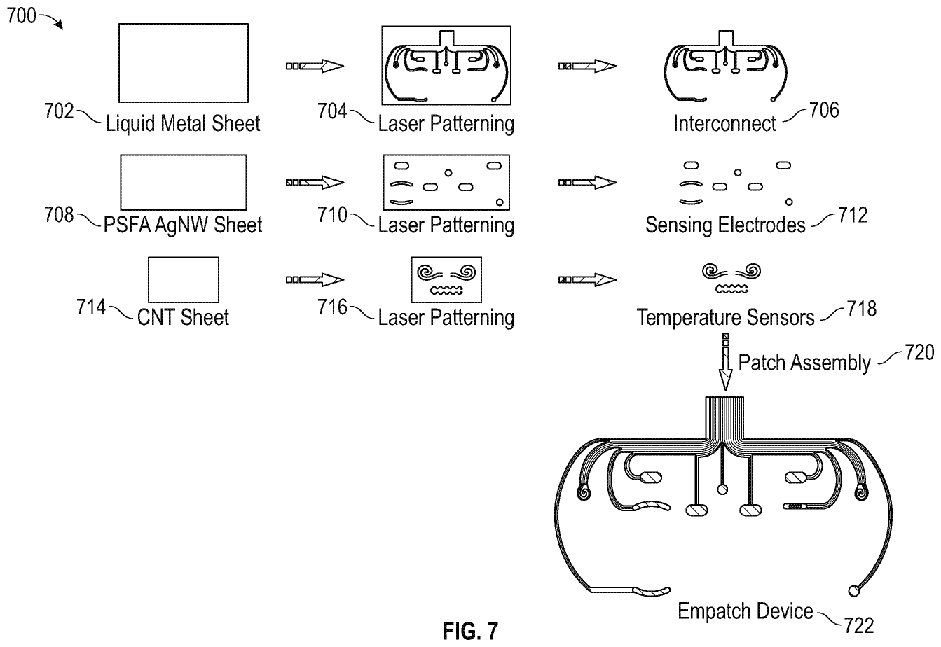

FIG. 7 is a diagram depicting an example wearable patch platform fabrication process, in accordance with various embodiments of the presently disclosed technology, shown generally at 700. The process shown in FIG. 7 illustrates one example multi-material manufacturing flow for forming stretchable interconnects and sensor elements and then assembling those elements into an integrated wearable patch platform capable of acquiring multimodal physiological data for use by the health profile generation and downstream inference components of the disclosed system. As with the hardware configurations described in connection with prior figures, the fabrication process illustrated here is exemplary, and one of ordinary skill in the art will appreciate that the disclosed system may be realized using a wide variety of wearable device fabrication approaches, materials, and assembly sequences. The EMPATCH platform described throughout this disclosure represents one particular instance of such a wearable device and is used here to illustrate a concrete example of how the disclosed sensing and stimulation hardware may be manufactured.

A first portion of the process may form stretchable electrical routing using a liquid metal sheet 702 that may be patterned by a first laser patterning step 704 to define a set of interconnects 706. The patterned liquid metal interconnects 706 may provide mechanically compliant conductive pathways that accommodate repeated stretching, bending, and twisting while maintaining electrical continuity, making them well-suited for routing signals between sensing regions, processing circuitry, and external communication interfaces within a soft wearable device. A second portion of the process may form skin-contact sensing structures using a PSFA silver nanowire sheet 708 of the type described in connection with FIGS. 3A-B, which may be patterned by a second laser patterning step 710 to define sensing electrodes 712. The porous and conductive architecture of the PSFA silver nanowire material makes these electrodes suited for long-duration, conformal skin-contact electrophysiological sensing of the type required to generate the multimodal physiological inputs consumed by the trained model. Additional sensing components may be formed using a carbon nanotube sheet 714 that may be patterned by a third laser patterning step 716 to define temperature sensors 718, which may provide the thermal measurement capability needed to capture the thermoregulatory physiological dimension of the multimodal sensing suite and to monitor the skin surface temperature response during delivery of thermal therapeutic stimulation.

A final portion of the process may include patch assembly 720, during which the interconnects 706, sensing electrodes 712, and temperature sensors 718 may be combined and integrated into a unified device structure. The assembled device 722 may represent an example of a wearable patch platform that acquires multimodal physiological data across the sensing modalities described herein and interfaces with the downstream wireless transmission, digital processing, and model inference components described in connection with FIGS. 4A and 4B to support real-time health profile generation, affective state inference, and closed-loop therapeutic intervention.

As used herein, the term multimodal physiological data may refer to data indicative of physiological state obtained from two or more sensing modalities and/or two or more data sources. Multimodal physiological data may include raw samples, processed signals, extracted features, and combinations of samples and features, and may be received as continuous streams, discrete batches, segmented windows, or event-triggered recordings. The number, type, and combination of modalities contributing to multimodal physiological data may vary across implementations, sessions, and subjects without departing from the scope of the presently disclosed technology.

As used herein, the term physiological signals may broadly refer to signals derived from a subject's body that are indicative of physiological state. Physiological signals may include electrical signals, thermal signals, electrodermal signals, optical signals, mechanical signals, biochemical signals, motion-derived signals, and derived quantities computed from any of the foregoing. Physiological signals may include raw waveforms as well as features such as spectral band powers, statistical descriptors, time-frequency representations, learned embeddings, and signal quality metrics, and any combination thereof may be used as inputs to the disclosed system.

As used herein, time-series data may refer to data associated with time. Time-series data may include ordered samples, irregularly spaced samples, windows, segments, epochs, or event-based measurements. Time-series data may be represented using timestamps, indices, sequence order, or inferred temporal coordinates, and the temporal organization of the data may vary across modalities and sources within the same implementation.

As used herein, missing portions may refer to an absence of expected data values in a time series at one or more times, channels, or modalities. Missing portions may arise from dropped samples, gaps in recording, intermittent sensor contact, duty cycling, buffering constraints, packet loss, or temporarily unavailable modalities. Missing portions may occur at the sample level, segment level, channel level, or device level, and the disclosed system may be designed to accommodate such absences without discarding the affected observation or requiring imputation prior to profile generation.

As used herein, irregular sampling may refer to sampling in which data points are not uniformly spaced in time. Irregular sampling may occur within a single modality or across modalities, and may be intentional, such as in power-saving duty-cycling strategies, or incidental, such as in variable transmission intervals and device scheduling variations. The domain-agnostic encoding and invertible mapping components of the disclosed system may accommodate irregular sampling by treating each incoming observation as an independently timestamped constraint on the subject profile rather than requiring uniform temporal spacing across inputs.

As used herein, temporal misalignment may refer to a condition in which samples from different modalities and/or data sources are not synchronized in time. Temporal misalignment may include fixed offsets, clock drift, variable transmission latency, different sampling frequencies, and different time bases across devices. Temporal misalignment may be corrected explicitly through preprocessing or handled implicitly through the profile-based integration and mapping approach described herein, which does not require that inputs from different modalities share a common temporal reference frame.

As used herein, a data source may refer to any device, system, repository, or process that provides physiological data. Data sources may include wearable devices, clinical instruments, mobile devices, remote sensors, ambient sensors, and stored datasets. Data sources may provide data in real time, near real time, or in offline batch mode, and the disclosed system may operate across any combination of these data delivery configurations.

As used herein, a subject profile may refer to a unified representation derived from multimodal physiological data that is maintained in a latent space and usable for one or more downstream processes. The subject profile may represent a time-varying latent state, time-indexed latent variables, or a distribution over latent variables. The subject profile may also include uncertainty measures, confidence measures, or quality measures derived from the density or reliability of constraints provided by the observed data, and these measures may inform how the system weights new observations relative to the accumulated profile state.

As used herein, latent space may refer to a representational space used by a trained model to encode multimodal physiological data into latent variables, embeddings, or state representations. Observation space may refer to a representational space that includes the multimodal physiological data as received, measured, derived, or preprocessed, and may include modality identifiers, device identifiers, timestamps, context signals, and quality indicators in addition to the physiological measurements themselves. The mapping between observation space and latent space may be learned during training and may be updated or adapted during deployment through the subject-specific weight adaptation mechanisms described herein.

As used herein, an invertible mapping may refer to a mapping between observation space and latent space for which an inverse mapping exists, enabling transformation from observation space to latent space and from latent space to observation space. In certain implementations, the mapping may be bijective within a modeled domain such that each observation corresponds to a unique latent representation and each latent representation corresponds to a unique observation. In other implementations, practical constraints such as quantization, clipping, or domain restrictions may yield an approximately invertible mapping while retaining the ability to compute meaningful inverse reconstructions for purposes of biomarker regeneration, missing data completion, and consistency enforcement.

As used herein, a trained model or trained machine-learning model may refer to a model having parameters established through a training process using data. The trained model may perform mapping between observation space and latent space, generation and/or updating of a subject profile, and production of downstream outputs including classification results, regression estimates, and reconstructed observations. Training may include supervised learning, self-supervised learning, semi-supervised learning, transfer learning, meta-learning, continual learning, and combinations thereof, and the trained model may be further adapted to individual subjects or datasets following initial training through the weight adaptation mechanisms described herein.

As used herein, a geometrically structured neural architecture may refer to a neural network that incorporates geometric constraints or structures into its learned transformations. A geometrically structured architecture may implement transformations on a manifold, including a Lie manifold of the type described in connection with FIGS. 2A-B. A manifold-based architecture may use structured operators such as projections, rotations, and scalings, and may use parameterizations that preserve invertibility and training stability, such as the matrix exponential parameterization of skew-symmetric generators described herein.

As used herein, an exponential map may refer to a mapping that relates elements of a local parameter space to points on a manifold, and may be used to parameterize model transformations in a manner that guarantees invertibility and geometric structure. In implementations that use Lie group transformations, exponential map parameterizations may produce reversible operators such as generalized rotations whose inverses are available analytically without requiring auxiliary storage structures. Multiple exponential-map-based transformations may be composed across layers or subspaces, and different subspaces may use different parameterizations to balance representational expressiveness and numerical stability.

As used herein, a shared representation may refer to a unified latent representation derived from multimodal physiological data, including the subject profile, an embedding, or other latent-state representation that aggregates information across modalities and/or sources. The shared representation may be designed to support a range of downstream tasks that may extend well beyond emotional state inference, including activity recognition, cognitive state estimation, alertness estimation, stress assessment, fatigue monitoring, anomaly detection, and physiological forecasting, as described further below.

The trained model may support integration of new observations over time through multiple approaches. One approach may update a subject profile by mapping each incoming observation into latent space and modifying the latent variables associated with a corresponding latent-space location. Another approach may update a subject profile by optimizing latent variables to satisfy accumulated observation constraints under the invertible mapping. Another approach may maintain a rolling profile state that is updated incrementally using state update rules that remain consistent with the invertibility of the underlying mapping. Any combination of these integration strategies may be employed depending on the computational resources available and the latency requirements of the application context.

Downstream use of the subject profile may be implemented through separate prediction heads, separate decoders, or shared layers followed by task-specific branches. The same profile may support both classification and regression tasks simultaneously, and may support generation of explanations by tracing from a downstream output back through the latent variables and to the observation-level constraints that most strongly determined the output. The system may also produce reconstructed observations, predicted future observations, or standardized physiological feature representations synthesized from the profile using the inverse mapping, any of which may be useful for validation, visualization, or integration with downstream clinical or consumer-facing interfaces.

As used herein, a controller may refer to one or more components that execute instructions to receive physiological data and generate outputs, and may include one or more processors and associated memory. The controller may be local to the wearable device, remote on a companion or cloud compute resource, distributed across multiple devices, or partitioned in any combination of these configurations. As used herein, operatively coupled may refer to a connection that enables functional interaction, including wired or wireless communication, direct or indirect electrical connection, and communication via intermediate components or network infrastructure.

As used herein, an action may refer to an output or operation produced based on a determined result, such as an inferred emotional state, a profile-based classification, or a regression estimate derived from the subject profile. Actions may include generating an alert, storing or transmitting data, controlling a device, scheduling a therapy session, modifying a user interface, or initiating delivery of a stimulus. Actions may be subject to safety constraints, rate limits, confidence thresholds, and user-defined preferences that govern when and how outputs from the system are acted upon.

As used herein, a stimulus device may refer to one or more devices that deliver an output perceptible by a subject, including audio, thermal, visual, immersive visual, haptic, and combinations thereof. As used herein, a stimulus may refer to the output delivered by such a device, which may be time-varying and may be personalized based on learned response patterns accumulated over prior interactions with the same subject. Stimuli may be delivered by a wearable device, by an external device, or by a coordinated combination of devices operating across multiple sensory modalities simultaneously.

In certain implementations, therapy selection and adaptation may incorporate priors and personalization mechanisms of the type described in connection with FIG. 5. A prior may represent population-level response expectations, clinician-defined constraints, or an initial user model initialized before subject-specific response data has been accumulated. Personalization may update user-specific weights or mappings based on measured physiological response and explicit or implicit user feedback, including self-reported ratings, engagement indicators, and inferred outcome measures derived from the evolving subject profile.

Planning and selection mechanisms may be implemented using heuristic search, policy rules, learned policies, or combinations thereof. For example, a therapy modality may be selected by evaluating candidate therapy states and choosing an action predicted to move the subject profile toward a desired region of an affective state space. Timing and repetition of therapy delivery may be managed using memory and habituation models, including decay curves of the type described in connection with FIG. 5 that represent the expected diminishing effect of a stimulus over time and that guide scheduling of reinforcement or modality transitions.

Compute deployment may vary across implementations and is not limiting on the scope of the presently disclosed technology. Processing and inference may execute fully on a wearable device, on a companion mobile device, on a local computing system, on a remote server, or across a distributed configuration combining any of the foregoing. Data may be streamed continuously, transmitted intermittently, buffered locally, or processed offline depending on the available communications infrastructure and the latency requirements of the application. Where desired, privacy-preserving implementations may transmit features or profile embeddings rather than raw physiological signals, and storage and transmission may be secured using encryption and access controls appropriate to the sensitivity of the physiological data involved.

Physical sensing implementations may also vary broadly and are not necessarily limiting on the scope of the presently disclosed technology. Wearable sensing may be provided by a bioelectronic patch of the type described herein, a smartwatch, a finger ring, a smart garment, a headband, a headset, or other form factors capable of acquiring physiological signals from the body. A skin-wearable patch may be skin-conformal and may include a sweat-permeable substrate, a porous conductive layer, skin-contact sensors, stretchable interconnect structures, and interfaces for wireless communications and power management. Other embodiments may omit a wearable patch and may operate on physiological data obtained from non-patch devices, stationary sensing platforms, clinical instruments, or stored datasets, without departing from the scope of the disclosed technology.

As used herein, a non-transitory computer-readable medium may refer to a tangible storage medium that stores instructions or data for execution by one or more processors, including semiconductor memory, magnetic storage, and optical storage, and excluding propagating signals per se. Method steps described in this disclosure may be embodied as instructions stored on such a medium and executed by one or more processors in any of the deployment configurations described above.

Although much of the present disclosure describes the disclosed system and methods in the context of emotional state determination, affective profiling, and emotion modulation therapy, it should be understood that the underlying architecture, including the invertible Lie manifold mapping, the domain-agnostic encoding of multimodal physiological inputs, the continuously updated subject profile, and the closed-loop sensing and intervention pipeline, may be applicable in a wide variety of contexts beyond emotional state management. For example, the disclosed system may be applied in clinical monitoring settings to track physiological state trajectories associated with pain, fatigue, cognitive load, or neurological conditions; in occupational and performance settings to assess operator readiness, vigilance, and workload; in rehabilitation settings to monitor recovery progress and adapt therapeutic exercise or stimulation protocols in response to measured physiological change; in sleep monitoring to characterize sleep stage transitions and deliver adaptive auditory or thermal stimuli to improve sleep quality; in human-computer interaction to enable affective and physiological state-aware interfaces that adapt to the user's condition in real time; and in research settings to characterize individual differences in physiological response across a broad range of experimental conditions. The scope of the presently disclosed technology is therefore not limited to any particular application domain, target state, or inference objective, and the system may be adapted to any context in which multimodal physiological time-series data is available and in which a continuously updated, invertible latent subject profile would be useful for inference, reconstruction, or closed-loop control.

Furthermore, while the present disclosure frequently references physical wearable sensing devices, including conformal skin patches, as the hardware platform through which physiological data is acquired, the disclosed system may also be implemented in connection with non-wearable or partially wearable sensing configurations, or in connection with stored or retrospectively collected physiological datasets, without requiring any or significant modification to the trained model, invertible mapping, or subject profile generation components. The system may receive physiological data from clinical bedside monitors, ambulatory recording systems, imaging-derived physiological estimates, contactless optical sensing platforms, or any other data acquisition modality capable of producing time-indexed physiological measurements. In implementations that do not involve a physical sensing device, the trained model may operate on previously recorded datasets, supporting retrospective profiling, population-level analysis, and transfer learning applications that leverage the same or similar latent representation and downstream inference capabilities described throughout this disclosure. The breadth of compatible data acquisition modalities and deployment configurations reflects the design objective of the disclosed architecture, which may be to provide a modality-agnostic, hardware-agnostic, and deployment-agnostic physiological intelligence platform whose core representational and inferential capabilities are determined by the geometry of the learned latent space rather than by the specifics of any particular sensing technology or hardware form factor.

The methods and systems described herein provide a practical technological solution to problems arising in computerized processing of real-world physiological time-series data, including data that is fragmented, irregularly sampled, temporally misaligned, and partially missing. Rather than being directed to an abstract idea of “analyzing information,” the disclosed subject matter improves the functioning of physiological data processing systems by enabling continuous profile construction, reconstruction, and updating without requiring the fixed synchronized input structures used by conventional approaches. These improvements are rooted in a specific computational architecture that defines an invertible mapping between an observation space and a latent space representation of a subject profile, which materially changes how missingness, misalignment, and heterogeneity are handled in software pipelines.

The disclosed techniques further recite specific computer processing steps and data transformations that go beyond mental processes and cannot practicably be performed in the human mind. For example, the trained model performs bidirectional transformations between observation space representations and latent space representations, supports profile generation and incremental profile updating as new data arrives, and can reconstruct missing portions by applying an inverse mapping. In implementations employing geometrically structured transformations, the model applies manifold-based operations, including exponential-map-based transformations and compositions of such transformations across layers or subspaces, which impose concrete computational constraints on internal representations and enable stable reversible computation across deep architectures.

The claimed subject matter also integrates, in certain implementations, with practical sensing and control environments, including wearable sensors and closed-loop stimulus delivery, and can be executed on-device, on a companion device, or in a distributed environment. This operational context reinforces that the disclosure is directed to technological improvements in sensor data acquisition and processing, real-time inference, and device control. The disclosed approach provides demonstrable improvements over conventional pipelines that require explicit synchronization, resampling, and imputation, and it provides a structured computational mechanism for robust profile construction and downstream decision support under typical wearable acquisition artifacts.

In summary, the detailed description sets forth model-centric techniques for generating and updating a unified subject profile from multimodal physiological time-series data using an invertible mapping between observation space and latent space, and for using that profile to support downstream inference and actions such as emotion-related monitoring and optional intervention. The disclosure also describes representative wearable implementations, digital processing pipelines, and therapy selection and adaptation workflows as examples that illustrate how the model can be deployed in real-world systems to improve robustness, interpretability, and continuous operation.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

The terms “approximately” and “substantially” are used to account for variations that may occur due to manufacturing tolerances, measurement inaccuracies, and other practical considerations in implementing the described technology. The term “approximately” refers to a value or range that is close to the stated value but allows for minor deviations, typically within ±10%, unless otherwise specified, that do not materially affect the function or purpose of the invention. Similarly, the term “substantially” is used to indicate that a particular feature, characteristic, or result is largely present or achieved, with allowable variations without deviating from the intended scope and function of the invention. These terms should be interpreted in a manner consistent with the understanding of a person skilled in the art.

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances or making or achieving performance better than that which can be achieved with other settings or parameters.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A computer-implemented method for monitoring and managing emotional states, the method comprising:
receiving multimodal physiological data associated with a subject from one or more data sources;
processing the multimodal physiological data with a trained model that defines an invertible mapping between (i) an observation space that comprises the multimodal physiological data and (ii) a latent space that represents a subject profile;
using the invertible mapping to generate the subject profile notwithstanding missing portions, irregular sampling, or temporal misalignment;
determining an emotional state of the subject based on the subject profile; and
causing performance of an action based on the determined emotional state.

2. The method of claim 1, wherein receiving the multimodal physiological data comprises receiving first multimodal physiological data to generate an initial subject profile, and thereafter receiving additional physiological data, wherein using the invertible mapping comprises mapping the additional multimodal physiological data into the latent space to update the subject profile without requiring temporal alignment between the additional multimodal physiological data and the first multimodal physiological data.

3. The method of claim 1, wherein the trained model comprises a geometrically structured neural architecture that performs transformations on a manifold.

4. The method of claim 3, wherein processing the multimodal physiological data comprises applying an exponential map to map between the latent space and the observation space.

5. The method of claim 4, wherein processing comprises applying a plurality of exponential-map-based transformations in respective layers or subspaces of the trained model.

6. The method of claim 1, wherein the invertible mapping is bijective between the observation space and the latent space.

7. The method of claim 1, wherein using the invertible mapping comprises reconstructing at least a portion of the multimodal physiological data corresponding to the missing portions.

8. The method of claim 1, wherein receiving the multimodal physiological data comprises receiving physiological data generated by a plurality of wearable devices associated with the subject.

9. The method of claim 1, wherein causing performance of the action comprises causing delivery of a stimulus to the subject, the stimulus comprising at least one of auditory stimulus, thermal stimulus, visual stimulus, or immersive visual stimulus.

10. The method of claim 1, wherein the multimodal physiological data comprises time-series data having at least one of: missing portions, irregular sampling, or temporal misalignment between modalities.

11. A computer-implemented method for monitoring and managing emotional states, the method comprising:
receiving multimodal physiological data associated with a subject from one or more data sources, wherein the multimodal physiological data comprises time-series data having at least one of: missing portions, irregular sampling, or temporal misalignment between modalities;
processing the multimodal physiological data with a trained model that defines an invertible mapping between (i) an observation space that comprises the multimodal physiological data and (ii) a latent space that represents a subject profile;
generating, with the trained model, an initial subject profile in the latent space based on a first portion of the multimodal physiological data;
after generating the initial subject profile, receiving additional multimodal physiological data associated with the subject, wherein the additional multimodal physiological data comprises at least one of (i) temporal misalignment with the first portion or (ii) missing portions;
mapping, with the trained model, the additional multimodal physiological data into the latent space to associate the additional multimodal physiological data with corresponding latent-space locations of the subject profile and to update the subject profile;
determining an emotional state of the subject based on the updated subject profile; and
causing performance of an action based on the determined emotional state.

12. The method of claim 11, wherein causing performance of the action comprises causing delivery of a stimulus to the subject based on the determined emotional state.

13. The method of claim 11, wherein the invertible mapping is bijective between the observation space and the latent space.

14. The method of claim 11, wherein the trained model comprises a geometrically structured neural architecture that represents transformations on a manifold.

15. The method of claim 14, wherein processing the multimodal physiological data comprises applying an exponential map to map between the latent space and the observation space.

16. The method of claim 15, wherein processing comprises applying a plurality of exponential-map-based transformations in respective layers or subspaces of the trained model.

17. The method of claim 11, wherein mapping the additional multimodal physiological data into the latent space comprises reconstructing at least a portion of physiological data corresponding to the missing portions.

18. The method of claim 11, wherein receiving the multimodal physiological data comprises receiving observed physiological data from a plurality of physiological sensing modalities of one or more sensing devices.

19. A computer-implemented method for generating and updating a unified subject profile from multimodal physiological data, the method comprising:
receiving multimodal physiological data associated with a subject from one or more data sources;
processing at least a portion of the multimodal physiological data with a trained model that defines an invertible mapping between (i) an observation space that comprises the multimodal physiological data and (ii) a latent space that represents a subject profile;
generating, using the invertible mapping, an initial subject profile in the latent space from a first portion of the multimodal physiological data;
after generating the initial subject profile, receiving additional multimodal physiological data associated with the subject;
applying the invertible mapping to associate the additional multimodal physiological data with corresponding latent-space locations and to update the subject profile without requiring temporal alignment of the additional multimodal physiological data to the first portion; and
outputting the updated subject profile for use in at least one downstream process.

20. The method of claim 19, wherein the invertible mapping is bijective between the observation space and the latent space.

21. The method of claim 19, wherein the trained model comprises a geometrically structured neural architecture that represents the invertible mapping as a composition of manifold-based transformations.

22. The method of claim 21, wherein processing comprises mapping between the latent space and the observation space using an exponential-map-based parameterization.

23. The method of claim 19, wherein the trained model comprises an invertible architecture including at least one volume-preserving transformation.

24. The method of claim 19, wherein applying the invertible mapping to associate the additional multimodal physiological data comprises applying an inverse of the invertible mapping to map between the latent space and the observation space.

25. The method of claim 19, wherein the trained model maps physiological data from a plurality of different modalities into a shared latent representation forming at least part of the subject profile.

26. The method of claim 19, wherein the multimodal physiological data comprises time-series data having at least one of: missing portions, irregular sampling, or temporal misalignment between modalities.

27. A system for monitoring and managing an emotional state, comprising:
one or more physiological sensors that generate multimodal physiological data associated with a subject;
a stimulus device that delivers stimulus to the subject; and
a controller operatively coupled to the one or more physiological sensors and the stimulus device, the controller comprising one or more processors and a non-transitory memory storing instructions that, when executed by the one or more processors, cause the controller to:
receive the multimodal physiological data;
process the multimodal physiological data with a trained model that defines an invertible mapping between (i) an observation space comprising multimodal physiological data and (ii) a latent space representing a subject profile;
generate the subject profile using the invertible mapping;
determine an emotional state of the subject based on the subject profile; and
control the stimulus device to deliver the stimulus based on the determined emotional state.

28. The system of claim 27, wherein the one or more physiological sensors are carried by a skin-conformal wearable patch comprising a sweat-permeable substrate.

29. The system of claim 28, wherein the sweat-permeable substrate comprises a porous layer defining a plurality of pores or apertures extending between a skin-facing surface of the wearable patch and an outward-facing surface of the wearable patch.

30. The system of claim 27, wherein the multimodal physiological data comprises time-series data having at least one of: missing portions, irregular sampling, or temporal misalignment between modalities.

31. The system of claim 27, wherein the wearable patch further comprises (i) conductive traces on the sweat-permeable substrate that electrically couple the one or more physiological sensors to an electrical interface, and (ii) a skin-adhesive layer on the skin-facing surface of the wearable patch.
